(19) United States
(12) Patent Application Publication
(10) Pub. No.: US 2023/0125432 A1
(43) Pub. Date: Apr. 27, 2023

(54) Systems, Devices, and Methods for Converting a Neutron Beam

(71) Applicant: TAE Life Sciences, LLC, Foothill Ranch, CA (US)

(72) Inventors: Charles Lee, Irvine, CA (US); Jedediah Styron, Wildomar, CA (US)

(21) Appl. No.: 17/974,331

(22) Filed: Oct. 26, 2022

Related U.S. Application Data
(60) Provisional application No. 63/272,670, filed on Oct. 27, 2021.
(60) Provisional application No. 63/331,290, filed on Apr. 15, 2022.

(51) Int. Cl. H05H 3/06; A61K 41/00; H05H 6/00; A61N 5/10

(52) U.S. Cl. CPC H05H 3/06; A61K 41/0095; H05H 6/00; A61N 5/1077

(57) ABSTRACT

Systems, devices, and methods for converting a raw neutron beam to a specified deliverable format having a targeted energy range, size, and direction are described. Embodiments of a neutron beam converter can include numerous regions based on location, function, dimension, and/or constituent material. The regions can include a central region, an intermediate region, a peripheral region, and a frontal region. Materials are also described.

### CROSS-REFERENCE TO RELATED APPLICATION

This application claims priority under 35 U.S.C. 119 to Provisional Application Nos. 63/272,670, filed Oct. 27, 2021, and 63/331,290, filed Apr. 15, 2022, both which are incorporated by reference.

### FIELD

The subject matter described herein relates generally to systems, devices, and methods for converting neutron beams from a raw form to a deliverable form.

### BACKGROUND

Boron neutron capture therapy (BNCT) is a modality of treatment of a variety of types of cancer, including some of the most difficult types. BNCT is a technique that selectively aims to treat tumor cells while sparing the normal cells using a boron compound. The boron compound allows for efficient uptake by a variety of cell types and selective drug accumulation at target sites, such as tumor cells. Boron loaded cells can be irradiated with neutrons (e.g., in the form of a neutron beam). The neutrons react with the boron to eradicate the tumor cells.

Neutron beams for BNCT can be generated through various techniques. One such technique involves irradiation of a suitable neutron generating target with a charged particle beam, such as a proton beam or deuteron beam. The charged particles react with nuclei in the target to emit a beam of raw neutrons that can be used for BNCT. The neutrons are raw in the sense that, immediately after generation in the target, a significant fraction of the neutrons may be propagating in various directions not directly towards the patient and may have energy levels too high or too low for administration to the patient. The raw neutrons may also be accompanied by undesired gamma radiation. Existing techniques for modifying the raw neutrons suffer from drawbacks, such as an insufficient ability to redirect neutrons in the desired direction, an insufficient ability to scatter neutrons to the desired energy range, and an insufficient ability to remove undesired photon and low energy neutron radiation. Accordingly, a need exists for improved systems, devices, and methods for neutron beam modification or conversion.

### SUMMARY

The subject matter described herein relates generally to systems, devices, and methods for converting a raw neutron beam to a specified deliverable format having a targeted energy range, size, and direction, as well as for removing undesirable non-neutronic radiation. Embodiments of a neutron beam converter (NBC) are described in an example context of a BNCT system configured to output a neutron beam in an epithermal energy range. The NBC can include numerous regions based on location, function, dimension, and/or constituent material. The regions can include a central region oriented along a beam axis between a beam input and a beam output of the NBC. The central region can be configured to primarily perform the function of scattering high energy neutrons down to the epithermal range, and achieving and/or maintaining a forward-facing beam (e.g., a beam propagating primarily in the direction of the patient). The central region can perform other functions such as redirection and neutron absorption. An intermediate region can be located laterally outside of the central region, and can function to redirect neutrons back into the central region while scattering towards or into the epithermal range, for output to the patient. The intermediate region can also absorb photons in the form of gamma radiation and can scatter neutrons propagating away from the beam axis down to epithermal and lower energy levels for more ready absorption. A peripheral region can be located laterally outside of the intermediate region and can function to scatter neutrons down to and into the epithermal and thermal energy ranges to absorb neutrons and to absorb photons in the form of gamma radiation. A frontal region can be located on the side of the NBC facing the patient and can also function to scatter neutrons down to and into the epithermal and thermal energy ranges, absorb neutrons, and absorb photons in the form of gamma radiation. Numerous example embodiments of NBC arrangements are disclosed that perform some or all of these functions in the central, intermediate, peripheral, and frontal regions. Numerous exemplary materials are disclosed with capabilities to perform one or more of these functions.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein and be protected by the accompanying claims. In no way should the features of the example embodiments be construed as limiting the appended claims, absent express recitation of those features in the claims.

### BRIEF DESCRIPTION OF DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is a schematic view depicting an example of a neutron beam system in accordance with the present disclosure.

FIG. 1B is a schematic view depicting an example of a neutron beam system for use in boron neutron capture therapy (BNCT).

FIG. 2A is a perspective view depicting an example of a neutron generating target.

FIG. 2B is a side view depicting an example of an assembly for housing a neutron generating target.

FIG. 2C is a cross-sectional view depicting an example of an assembly for housing a neutron generating target.

FIG. 3A is a cross-sectional view depicting an example embodiment of a neutron beam converter.

FIG. 3B is a rear perspective view depicting an example embodiment of a neutron beam converter.

FIGS. 4A-4K are cross-sectional views depicting example embodiments of central regions of a neutron beam converter.

FIGS. 5A-5H are cross-sectional views depicting example embodiments of stacked configurations for central regions of a neutron beam converter.

FIGS. 6A-6B are cross-sectional views depicting example embodiments of intermediate regions of a neutron beam converter.

FIGS. 7A-7B are cross-sectional views depicting example embodiments of peripheral regions of a neutron beam converter.

FIGS. 8A-8B are cross-sectional views depicting example embodiments of frontal regions of a neutron beam converter.

FIGS. 9A-9C are cross-sectional views depicting example embodiments of a first configuration of a neutron beam converter.

FIGS. 10A-10C are cross-sectional views depicting example embodiments of a second configuration of a neutron beam converter.

FIGS. 11A-11C are cross-sectional views depicting example embodiments of a third configuration of a neutron beam converter.

FIGS. 12-13 are perspective view depicting portions of example embodiments of a neutron beam converter having various transitional surface shapes.

FIGS. 14A-14Q are graphs of cross-section versus neutron energy for materials of various types suitable for use within an example embodiment of a neutron beam converter.

### DETAILED DESCRIPTION

This disclosure is not limited to the particular embodiments described as such may, of course, vary. The terminology used herein is for the purpose of describing particular embodiments only and is not intended to be limiting since the scope of the present disclosure will be limited only by the appended claims.

The term “particle” is used broadly herein and, unless otherwise limited, can be used to describe an electron, a proton (or H+ ion), or a neutron, as well as a species having more than one electron, proton, and/or neutron (e.g., other ions, atoms, and molecules).

Example embodiments of systems, devices, and methods are described herein for neutron beam conversion, which can be used in combination with a neutron beam system (e.g., including a reactor or a particle accelerator). The embodiments described herein can be used with any type of neutron beam system in which neutron beam conversion or modification is desired. Embodiments herein can be used in numerous applications, an example of which is a neutron beam system for generation of a neutron beam for use in boron neutron capture therapy (BNCT). BNCT uses a beam of epithermal neutrons (e.g., with an energy spectrum between one electronvolt (eV) and thirty kiloelectronvolts (keV)) for cancer treatment. In BNCT, the neutrons can be generated from nuclear reactions of charged particles (e.g., a proton beam) colliding with either a beryllium or a lithium target device. The generated neutron beam has a broad range of energies and is emitted in a variety of directions. Thus the target can be contained within a larger neutron beam converter (NBC) that functions to convert the generated neutron beam into a primarily forward directed beam within the desired epithermal energy range, which is then output to the patient.

The example embodiments of neutron beam converters described herein are not intended to be viewed in isolation from each other. All features, elements, components, and functions described with respect to any converter embodiment provided herein are intended to be freely combinable and substitutable with those from any other converter embodiment. If a certain feature, element, component, and function is described with respect to only one converter embodiment, then that that feature, element, component, and function can be used with every other converter embodiment described herein unless explicitly stated otherwise. This paragraph therefore serves as antecedent basis and written support for the introduction of claims, at any time, that combine features, elements, components, and functions from different converter embodiments, or that substitute features, elements, components, and functions from one converter embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

### Examples of Neutron Beam Systems

For ease of description, the embodiments described herein will be done so in the context of generating a neutron beam for use in BNCT, although the embodiments are not limited to such. The embodiments can be applied to other applications that generate significant neutron radiation, even those outside of BNCT applications utilizing different energy ranges.

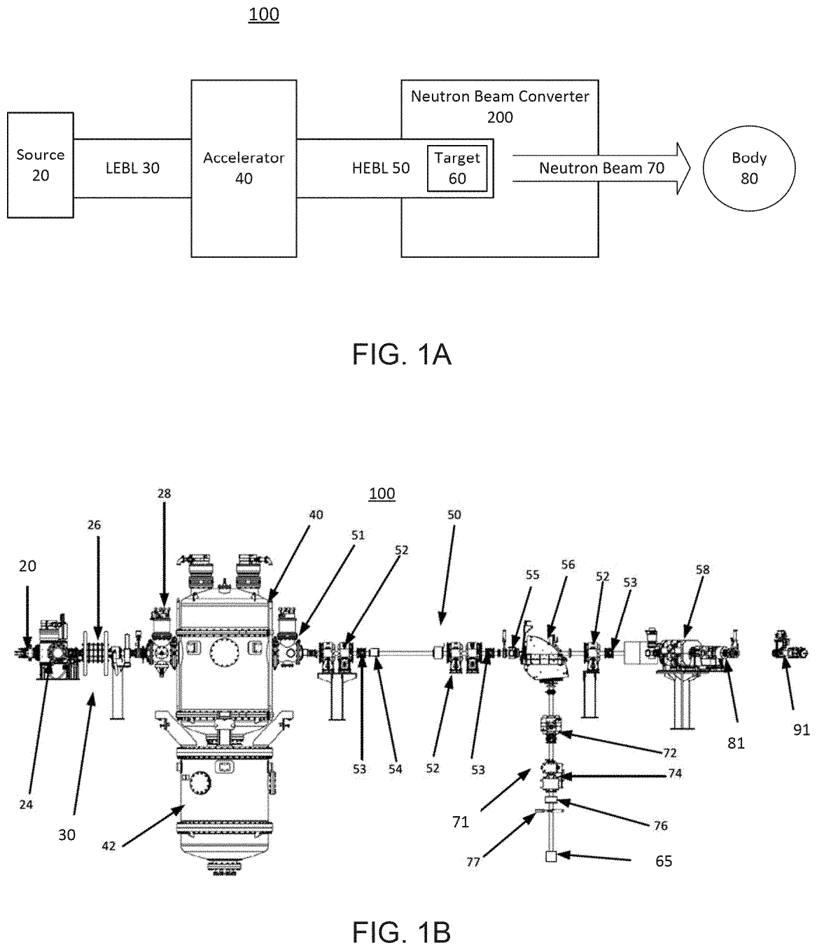

FIG. 1A illustrates a schematic view of an example embodiment of a system 100 for use in BNCT, in accordance with the present disclosure. System 100 is configured to create a charged particle beam and propagate it to a target 60 to generate a neutron beam 70 that is directed towards a patient body 80 to be irradiated. Beam system 100 includes a charged particle source 20, a low-energy beamline (LEBL) 30, an accelerator 40, and a high-energy beamline (HEBL) 50. Source 20 is configured to generate the charged particle beam, which is output to LEBL 30. LEBL 30 is configured to transport the beam from source 20 to accelerator 40. Accelerator 40 is configured to accelerate the charged particle beam to a higher energy. HEBL 50 extends from the accelerator 40 to target 60 housed within a target assembly portion of HEBL 50. HEBL 50 transfers the charged particle beam from an output of accelerator 40 to target 60, where it is converted to neutron beam 70.

Neutron beam converter (NBC) 200 is positioned close to and around target 60 to perform various functions on the neutrons of beam 70 emanating from target 60. These functions include reducing the energy of generated neutrons from energies above the desired range to within the desired range, focusing the generated neutrons in a forward-facing direction towards the patient, removing generated neutrons that are outside the desired range, and removing other radiation byproducts (e.g., such as photons) at energy levels that are undesirable. The desired neutron energy range can vary based on the application. For the BNCT applications described herein, the desired energy range can be, for example, one eV to ten keV, or one eV to thirty keV, with the neutron distribution peaking near the upper end of the desired range. For example, a one eV to 30 keV beam can be configured to output at least 90% of the neutrons in that energy range with a peak neutron distribution and an average energy between 10 keV and 30 keV. By way of another example, a one eV to ten keV beam can be configured to output at least 90% of the neutrons in that energy range with a peak neutron distribution and an average energy between three keV and 10 keV. For convenience these ranges will be described as epithermal energy ranges. Neutrons at energies beneath these ranges will be referred to as thermal neutrons (e.g., beneath one eV), and those above these ranges will be referred to as fast neutrons (e.g., above 30 keV).

FIG. 1B is a schematic view illustrating an example embodiment of beam system 100 configured as a neutron beam system for use in BNCT. Beam system 100 includes a pre-accelerator system 26 forming at least a portion of LEBL 30, where pre-accelerator system 26 serves as a charged particle beam injector. System 100 includes a high voltage (HV) tandem accelerator 40 coupled to LEBL 30, and HEBL 50 extending from tandem accelerator 40 to a target 60, as described with reference to FIG. 1A.

LEBL 30 transfers a negative ion beam (e.g., H− ions) from ion source 20, through pre-accelerator 26 which boosts the energy level of the ion beam and converges the ion beam, to an input (e.g., an input aperture) of accelerator 40. Accelerator 40 is powered by a high voltage power supply 42 coupled thereto. Accelerator 40 includes a vacuum tank, a charge-exchange tube, accelerating electrodes, and a high voltage feedthrough. Accelerator 40 can, in some implementations, accelerate a hydrogen ion beam to produce a proton beam with an energy generally equal to twice the voltage applied to the accelerating electrodes positioned within accelerator 40. The energy level of the proton beam can be achieved by accelerating the beam of negative hydrogen ions from the input of accelerator 40 to the innermost high-potential electrode, stripping two electrons from each ion, and then accelerating the resulting protons downstream by the same voltages encountered in reverse order.

HEBL 50 can transfer the proton beam from the output of accelerator 40 to the neutron-generating target 60 positioned at the end of a branch 71 of the beamline extending into a patient treatment room. Beam system 100 can be configured to direct the proton beam to one or more targets 60 and associated target areas. In some implementations, HEBL 50 includes multiple (e.g., three) branches 71, 81, and 91 configured to extend to multiple different patient treatment rooms, with each branch terminating in a target 60 and NBC 200. HEBL 50 includes a pumping chamber 51, quadrupole magnets 52 and 72 to prevent de-focusing of the beam, dipole or bending magnets 56 and 58 to steer the beam towards one or more targets, beam correctors 53, diagnostics such as current monitors 54 and 76, a fast beam position monitor 55 section, and a scanning magnet 74 for branch 71. Branches 81 and 91 can contain components similar to branch 71.

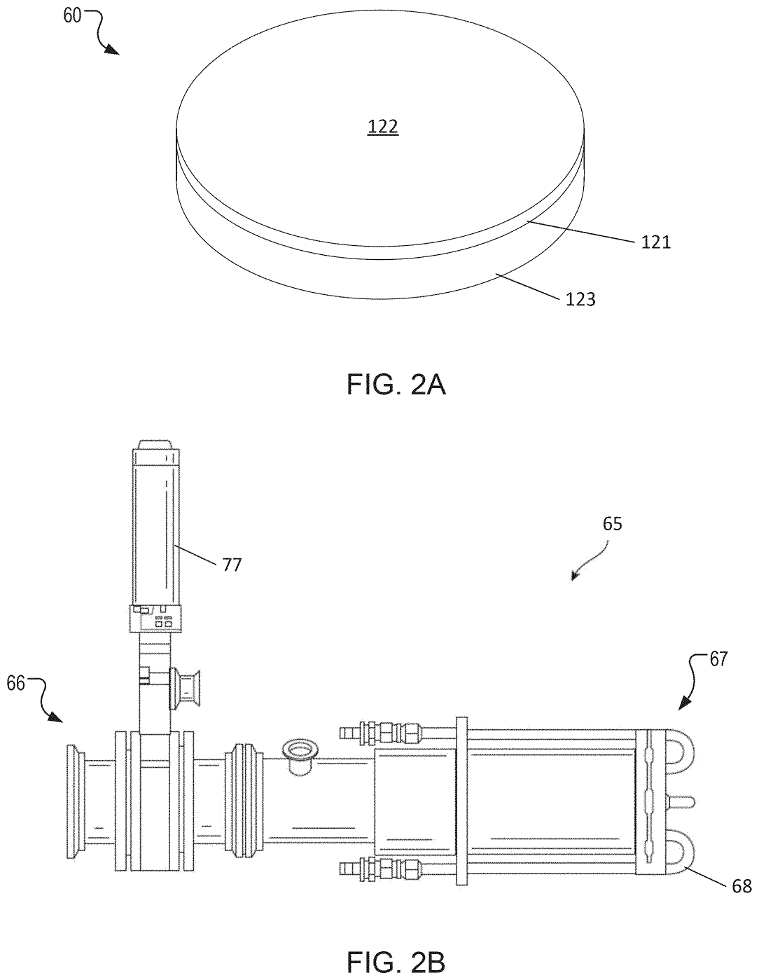

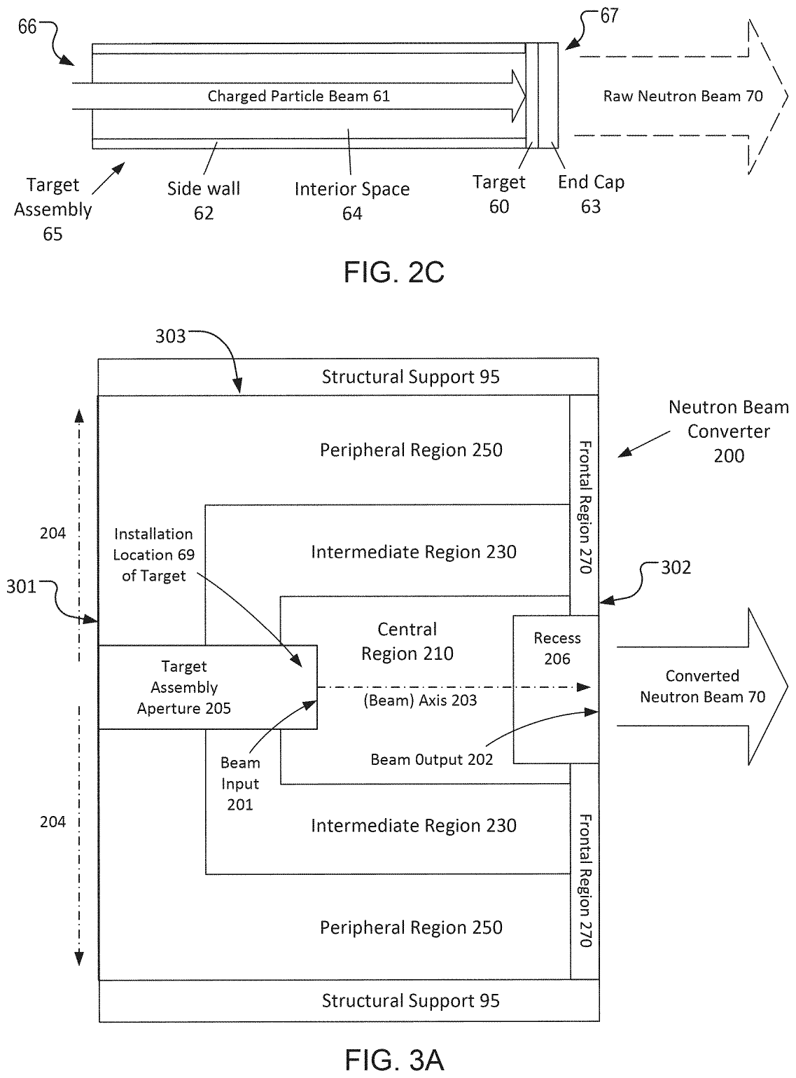

The design of HEBL 50 depends on the configuration of the treatment facility (e.g., a single-story treatment facility, a two-story treatment facility, and the like). The beam can be delivered to target 60 (e.g., positioned near a treatment room having a patient 80) with the use of the bending magnet 56. Quadrupole magnets 72 can be included to then focus the beam to a certain size at target 60. The beam can pass one or more scanning magnets 74, which provide lateral movement of the beam onto the target surface in a desired pattern (e.g., spiral, curved, stepped in rows and columns, combinations thereof, and others). The beam lateral movement can enable generation of smooth and even time-averaged distribution of the proton beam on the target 60, preventing overheating of the target and making the particle (e.g., neutron) generation as uniform as possible within the target (e.g., neutron generating layer 121 of FIG. 2A).

Scanning magnets 74 can be configured to direct the beam to a current monitor 76, which measures beam current. The beam current value can be used to operate a safety interlock. The target assembly 65 containing target 60 can be physically separated from the high-energy beamline volume with a valve 77. A function of valve 77 is to separate the vacuum volume of the beamline from the target 60 during removal of a used target and loading of a new target. In some implementations, instead of being bent by 90 degrees by a bending magnet 56, the beam can be directed straight to one or more quadrupole magnets 52 located in the horizontal beamline. The beam could be bent by another bending magnet 58 to a preset angle, depending on a setting requirement (e.g., location of a patient or a room configuration). In some implementations, bending magnet 58 can be arranged at a split in the beamline and can be configured to direct the beam in one of two directions for two different treatment rooms located on the same floor of a medical facility.

System 100 as described with respect to FIG. 1B is one example of different configurations that can be used to generate charged particle and neutron beams. Different configurations of system 100 can utilize accelerators other than electrostatic tandem accelerators, and can utilize targets that are either fixed or rotating. The embodiments of NBC 200 described herein are not limited to use with any one type of neutron beam generating system.

FIG. 2A is a perspective view of an example embodiment of a target 60. In this embodiment target 60 has a neutron generating layer 121 with a charged particle receiving face surface 122. Neutron generating layer 121 is positioned on or in proximity to a substrate 123. In some cases layer 121 is covered with one or more other layers for protection. Layer 121 can also have one or more underlying layers between layer 121 and substrate 123, e.g., to resist blister formation. A charged particle beam, such as a proton beam, incident upon face 122, passes into target 60 and causes layer 121 to undergo a reaction that generates neutrons. This is the Li-7(p,n)Be-7 nuclear reaction in the case where neutron generating layer 121 is composed of lithium-7. Neutron generating layer 121 can alternatively be beryllium-9, and neutrons can be generated with a proton beam (Be9(p,n)B9) or deuteron beam (Be9(d,n)B10) at different energies. Substrate 123 can be a material with excellent thermal conductivity, such as copper or aluminum, to assist in removal of the heat generated by the reactions.

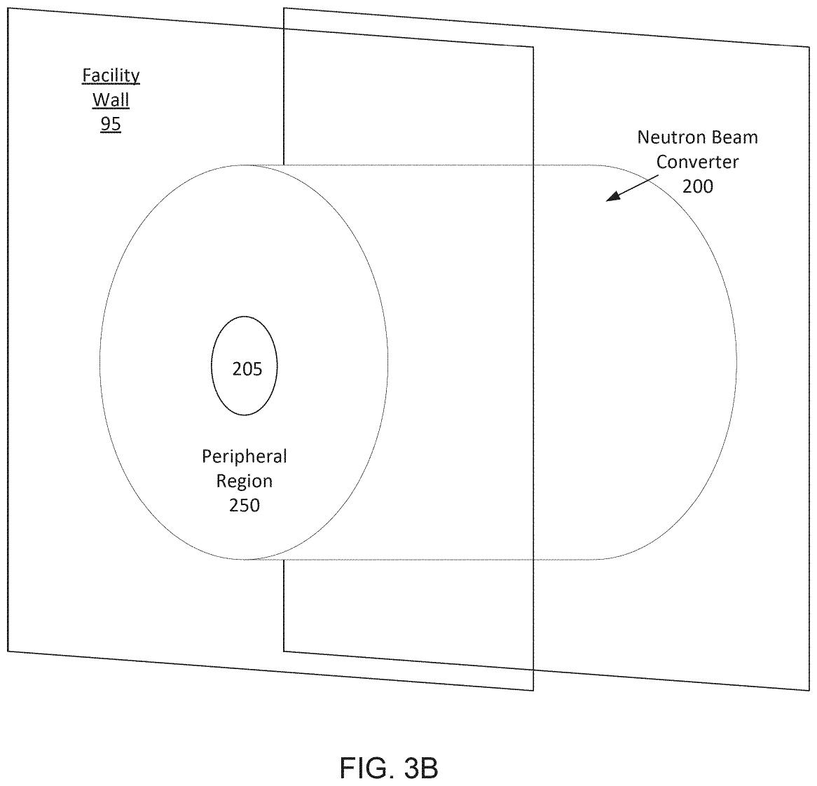

FIG. 2B is a side view of an example embodiment of a target assembly 65 that can form a terminal portion of HEBL 50. Target 60 (not shown) can be contained within assembly 65 at or near end 67. The charged particle beam enters assembly 65 at end 66 and travels to the opposite end 67 where it impacts target 60. Various cooling channels 68 are routed to and from end 67 for the insertion and removal of coolant used to regulate the temperature of target 60 during use. Numerous sensors can be included to monitor temperature and radioactivity of and around assembly 65. Also shown is valve 77 in the example form of a gate valve. End 67 of assembly 65 is inserted into an aperture 205 (FIG. 3A) within NBC 200 where it remains during BNCT procedures. Assembly 65 (with target 60) can be removed from NBC 200 and disposed of upon reaching the end of its usable lifetime, at which point a new assembly 65 and target 60 can be inserted into NBC 200.

FIG. 2C is a cross-sectional view of an example embodiment of target assembly 65 omitting components such as valve 77, coolant channels and sensor connections for clarity. A sidewall 62 has a tubular shape and contains an interior space 64 at a vacuum or near vacuum level. Target 60 is positioned at end 67 and held in place by end cap 63. Variations of this construction are possible, such as with target 60 surrounded by sidewall 62. Charged particle beam 61 is directed through interior space 64 and scanned across target 60 by scanning magnet 74 located upstream on HEBL 50 (not shown). Neutrons produced by target 60 will be emitted at some level in virtually all directions from target 60, but the majority of the neutrons will be emitted in a disperse but generally forward directed path. This is depicted here as neutron beam 70 in raw form.

### Example Embodiments of Neutron Beam Converters

FIG. 3A is a cross-sectional view of an example embodiment of neutron beam converter (NBC) 200. NBC 200 includes a target assembly aperture 205 that is configured to receive target assembly 65 (not shown). With an assembly configuration like that described with respect to the embodiment of FIG. 2B, upon installation of assembly 65 within aperture 205, target 60 would be positioned in target installation location 69. A generally close fit can be desirable although some amount of gap will be present between assembly 65 and the surrounding walls of NBC 200 in order to permit, e.g., routing of coolant channels and periodic exchanges of assembly 65.

NBC 200 is configured to have a beam input 201 adjacent to, or in close proximity with, target installation location 69. In some embodiments, the distance between input 201 and location 69 is 10 to 60 centimeters (cm), more preferably 25 to 40 cm. NBC 200 has a beam output 202 downstream of the generated neutron flow, which is located in proximity with recess 206. An axis 203 extends from input 201 to output 202 and, in this embodiment, is located generally centrally within NBC 200. For convenience, the position of elements within NBC 200 will be referred to with respect to axis 203 and lateral directions 204, which are perpendicular to axis 203. The terms upstream and downstream are referenced with respect to charged particle beam flow into the target and subsequent neutron flow, both of which proceed in the general direction from left to right on FIG. 3A (e.g., from input 201 to output 202 along axis 203). For example, aperture 205 is axially upstream of recess 206.

NBC 200 has a rear (upstream-most) face or side 301, a front (downstream-most) face or side 302, and a lateral face or side 303. NBC 200 includes four general regions: central region 210 which is traversed by axis 203; intermediate region 230; peripheral region 250; and frontal region 270. In this embodiment, central region 210 has a generally cylindrical shape. Intermediate region 230 also has a generally cylindrical shape and surrounds the lateral sides and the upstream side of central region 210. Peripheral region 250 has a generally cylindrical shape as well and surrounds the lateral sides and the upstream side of intermediate region 230. Regions 210, 230, and 250 can be configured to form generally concentric housings about axis 203, with the housings being in concentric cylindrical or pseudo-cylindrical multi-sided shapes.

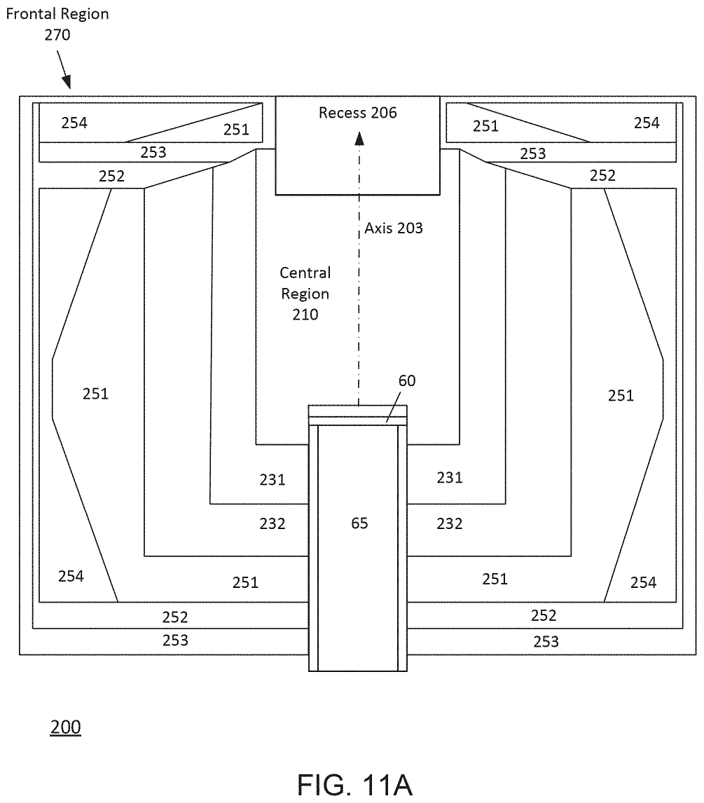

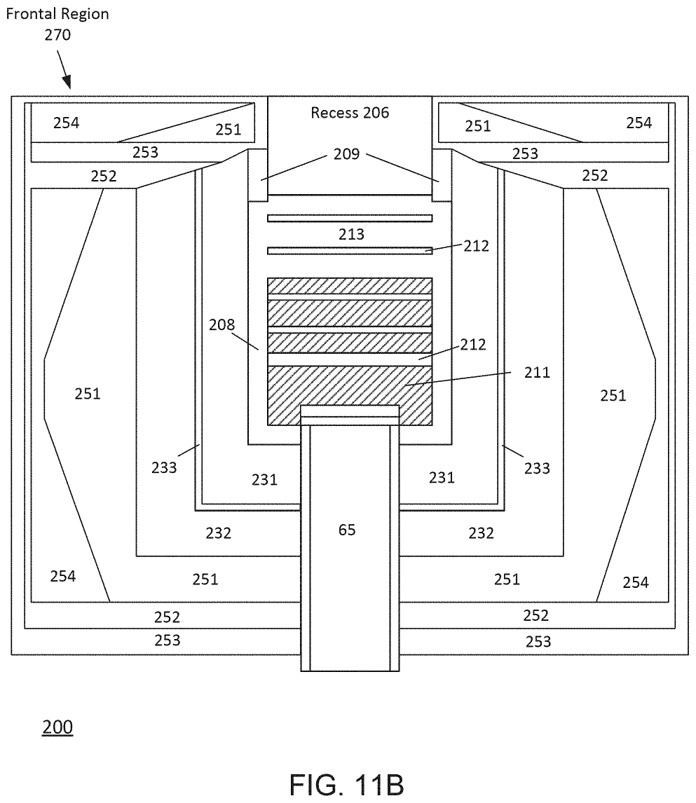

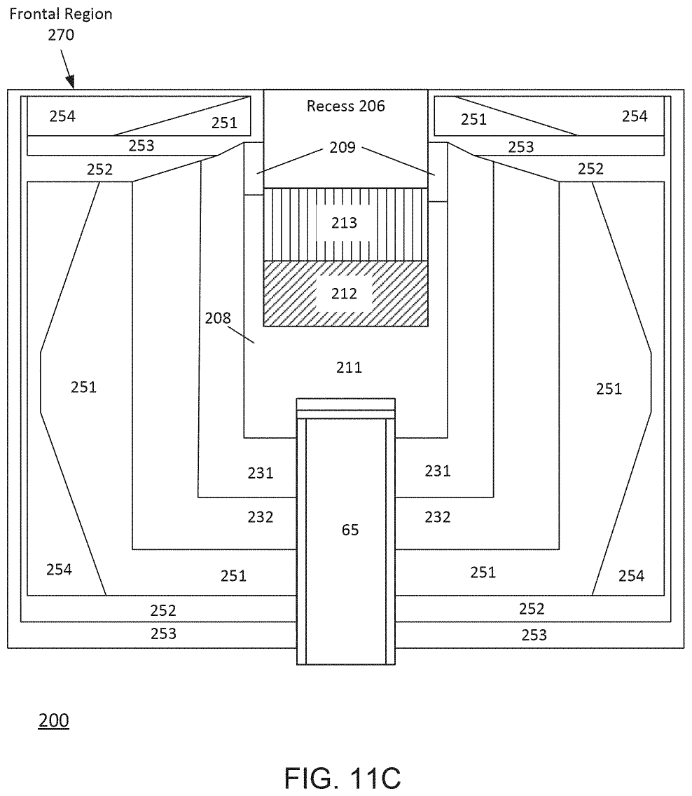

The various cylindrical shapes enable conditioning of neutrons emanating from target 60 on all lateral sides of the beam axis. The shape can be symmetrical in both the axial and lateral profiles, or asymmetric in either or both profiles. An asymmetric axial profile can have a variable diameter that increases or decreases progressing in an upstream-to-downstream direction (see section 251 of FIGS. 11A-11C). An asymmetric lateral profile can have a variable diameter when viewed in lateral cross-section (e.g., an elliptical cross-section).

Frontal region 270 is present across the downstream sides of regions 210, 230, and 250, although not necessarily in contact with each region. Recess 206 can be filled with a vacuum or ambient gas to aid in forming neutron beam 70 in the forward direction. NBC 200 can be mounted within a structural support 95 that maintains NBC 200 in position with respect to the upstream neutron beam system 100 and the downstream patient treatment room. FIG. 3B depicts a perspective view of an example embodiment of NBC 200 from the upstream side. In this embodiment, structural support 95 is a concrete BNCT facility wall.

Each of regions 210, 230, 250, and 270 are configured to perform a different array of functions on the neutrons and other particles generated by system 100. Central region 210 has the primary function of scattering fast neutrons emitted by target 60 in the forward direction without significant directional change, and without absorbing a substantial amount of neutrons. The fast neutrons are preferably scattered such that their energies descend into and remain within the target range (e.g., epithermal energies) until output from NBC 200. This preferably occurs without simultaneously scattering a significant number of neutrons down out of the target range.

The amount and type of material within central region 210, in any given direction, is proportional to the change in energy required to scatter and reduce the most probable neutron energy emitted in that direction. For example, more material is required in the forward direction since the neutrons emitted from target 60 in that direction are more energetic than those traveling in the backward direction.

Intermediate region 230 functions to redirect neutrons back towards central region 210 to conserve those neutrons and allow them to be utilized in beam 70, but in such a way that minimal energy loss and absorption occurs. Region 230 also scatters fast neutrons emitted at large forward angles from target 60 that laterally traversed central region 210, and provides photon shielding for prompt gamma rays produced by neutron captures in both central region 210 and peripheral region 250.

Peripheral region 250 functions to remove neutrons of all energies as it is unlikely those neutrons can be redirected back into central region 210. This is accomplished by further scattering the neutrons to thermal or near thermal energy levels for easier absorption by region 250. Region 250 also provides photon shielding to reduce the number of photons produced in the inner area of NBC 200 from entering the facility.

The effectiveness of a material to scatter and reduce the energy of a neutron is inversely proportional to its atomic mass number A, governed by the number of protons and neutrons in the nucleus. The maximum and average fractional energy loss for a material is approximated by the following equation:

$${( \frac{Δ E}{{E}_{i n i t i a l}} )}_{max} = ( 1 - α ) {and ( \frac{Δ E}{{E}_{i n i t i a l}} )}_{a v g} = \frac{1 - α}{2} , where α = {( \frac{A - 1}{A + 1} )}^{2}$$

As an example, a neutron interacting with a hydrogen nucleus (A≈1) will lose on average half of the initial energy per collision, and can lose up to all its energy in that single collision. For comparison, a neutron interacting with a nucleus of lead-208 (Pb-208, A≈208) or beryllium-9 (Be-9, A≈9) will result in ≈1% and 18% average energy losses, respectively, with maximum energy losses of ≈2% and 36%. This makes hydrogen 50 times more effective than lead and 2.5 times better than beryllium as a scattering material. For BNCT, however, a more effective scattering material is not necessarily more useful since a small value of a means a few collisions is sufficient to push the neutron below the epithermal energy range. The α value must be balanced with the other functions of NBC 200.

The number of collisions, on average, a neutron must undergo to down scatter from some incident neutron energy to a specific energy is based on another parameter ζ, which is given by the relationship:

$$ζ \approx \frac{2}{A + \frac{2}{3}} ( A > 1 ) ; ζ = 1 ( A = 1 )$$

The number of collisions, N, can be approximated using the following relationship:

$$N = - \frac{1}{ζ} ln ( \frac{{E}_{i n i t i a l}}{{E}_{final}} )$$

From this equation it can be shown that on average 24 times more collisions are needed in lead than in beryllium to reduce the incident neutron energy by half.

Each material, in addition to mass, has an energy dependent probability that a neutron will interact within the material, called the total macroscopic cross-section (Σₜₒₜ), or probability of collision per unit distance (e.g., 1/unit distance). At energies above thermal and below the first nuclear state the cross section is generally constant, and is referred to herein as the non-resonant region. Higher energies are referred to as the resonant region (defined by the discrete nuclear states) where peaks and valleys for the cross section are present, with the peaks corresponding to the energy of each discrete nuclear state and the valleys corresponding to the energy regions between resonances. If a neutron encounters a material with a large cross section resonance at the energy of the neutron, the probability of scattering to a lower energy is high. Conversely, relatively few scattering events will occur for neutron energies in a cross-section valley.

The type and amount of material present within each region can be selected to perform these various functions. Materials adept at scattering neutrons, classified generally herein as “S” materials, can be further classified as having substantial cross-sections for scattering from fast to epithermal energies (“Fast-S”), from fast and epithermal energies to lower energies (“Epi-S”), and as having substantial cross-sections in non-resonant regions (“NR-S”) for scattering fast and/or epithermal neutrons. Different scattering materials, regardless of class, can be indicated by a numeric suffix (e.g., S1, S2, and S3). Materials adept at absorbing thermal neutrons are generally referred to herein as “Ab” materials. Materials adept at redirecting neutrons are generally referred to herein as “R” materials.

### Example Fast-S Materials

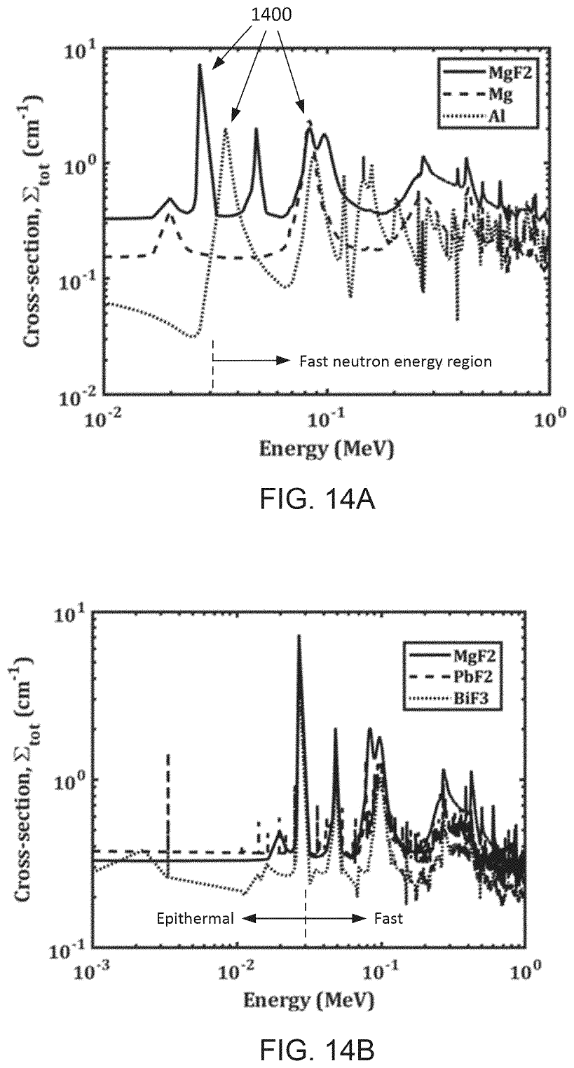

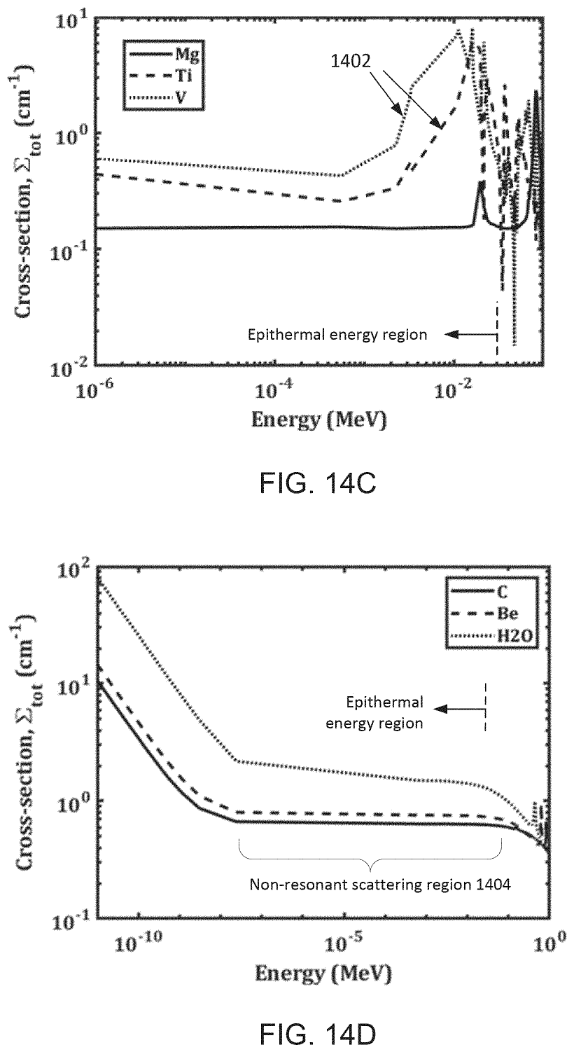

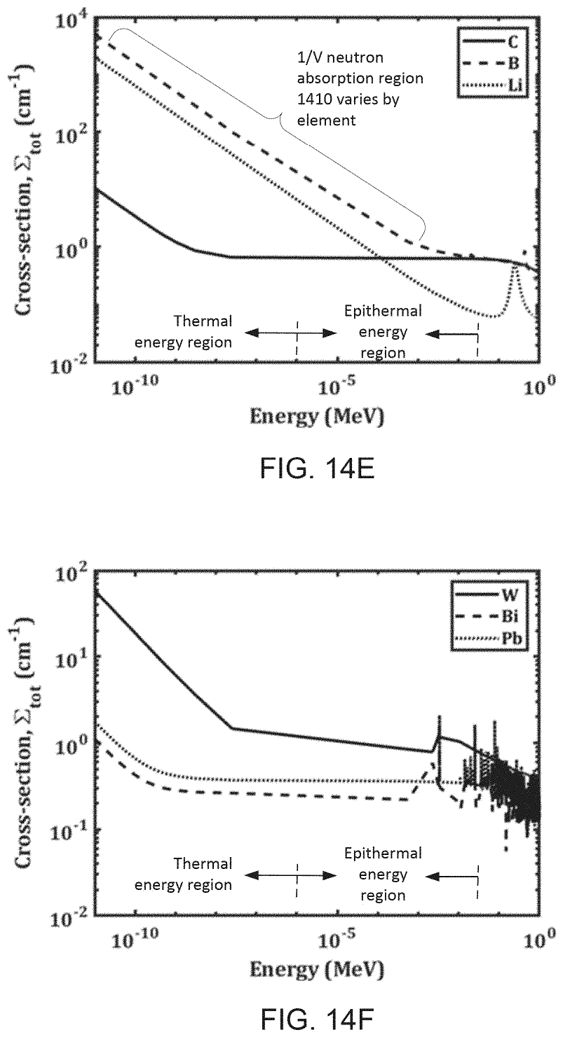

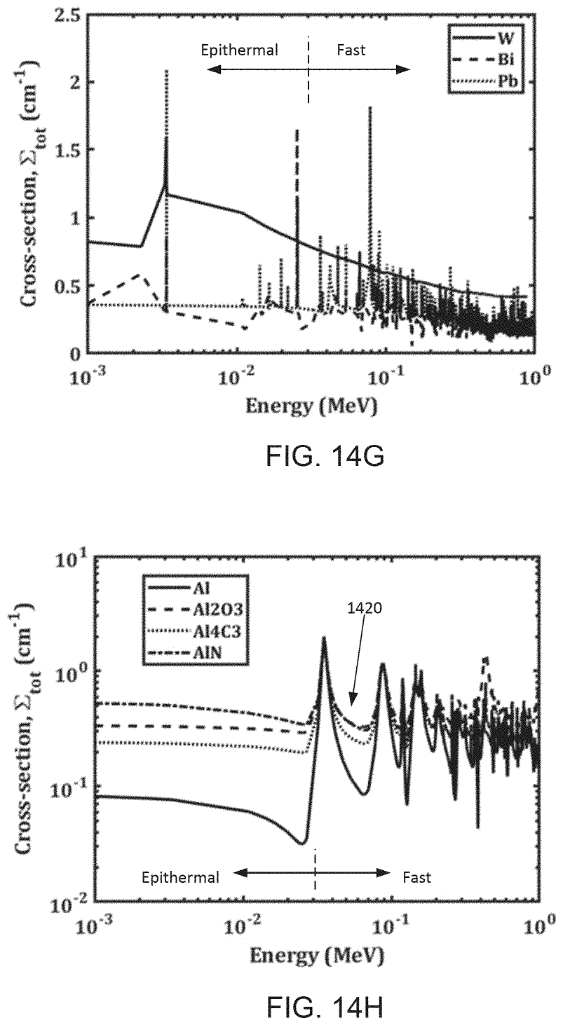

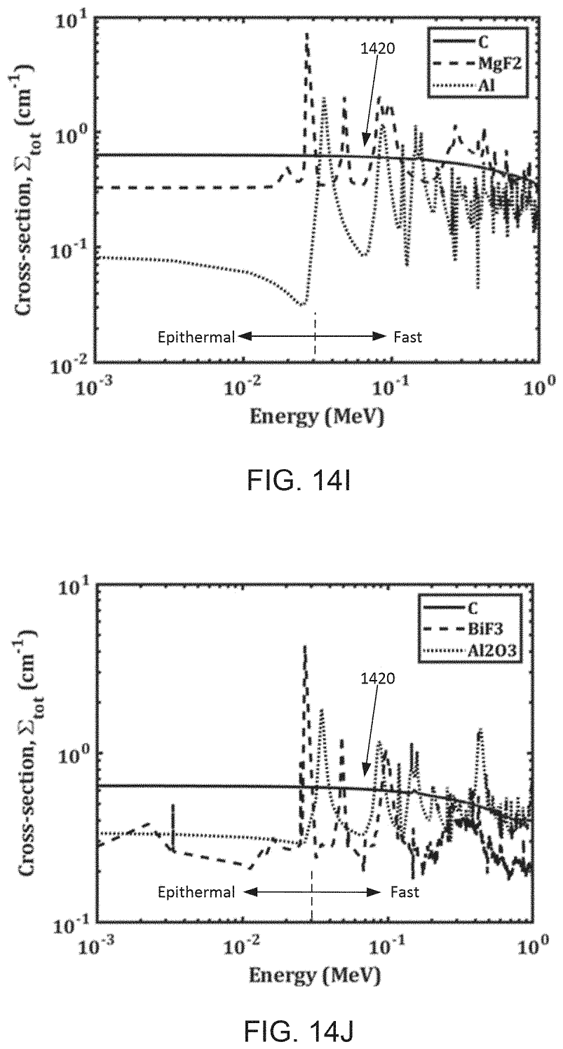

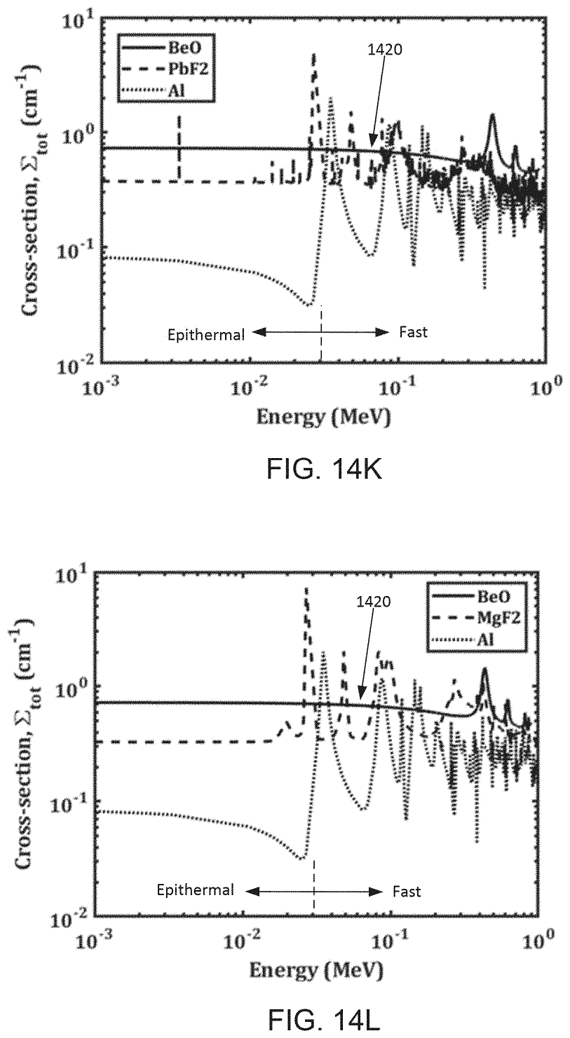

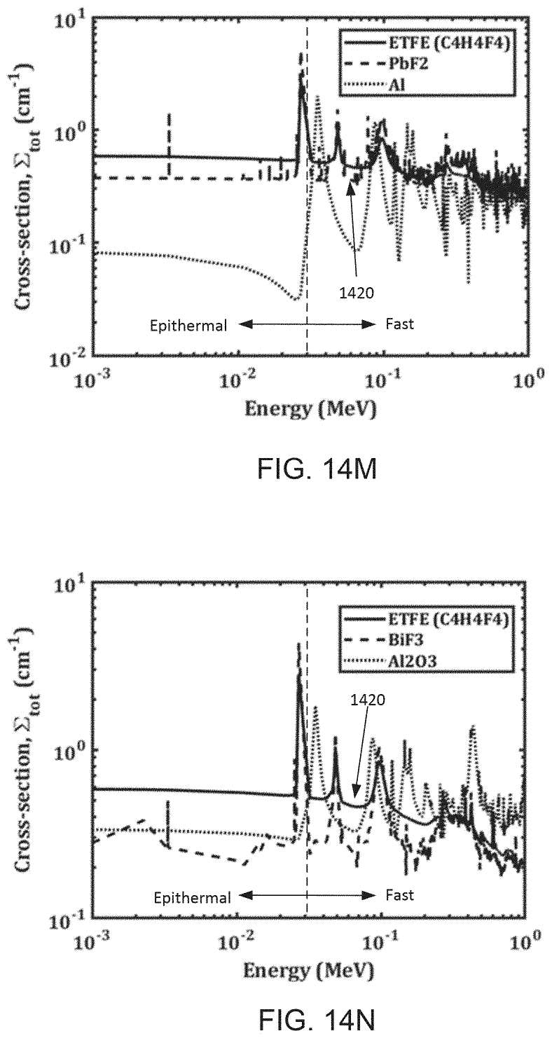

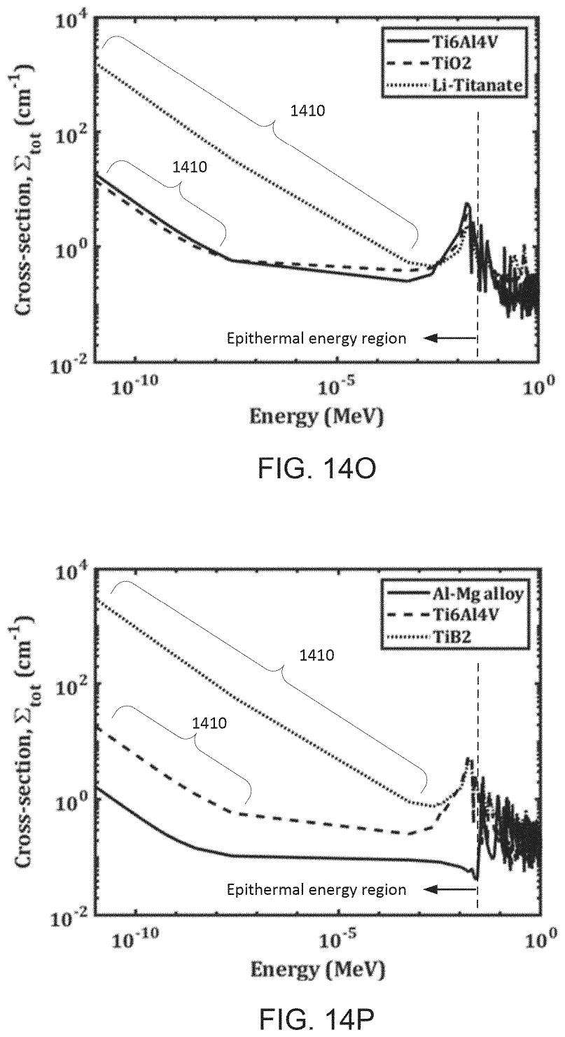

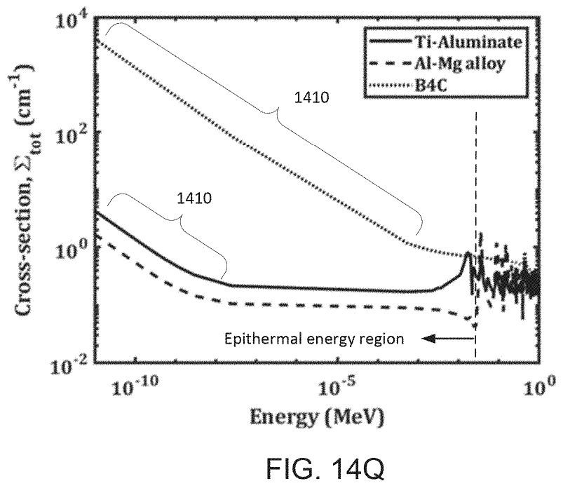

Particularly useful Fast-S elements can have an atomic number (Z) of nine or greater, examples of which are magnesium, fluorine, and aluminum. FIG. 14A is a graph depicting cross-section versus energy for magnesium fluoride (MgF₂), magnesium, and aluminum. As can be seen fluorine (˜27 keV) and aluminum (˜33 keV) have significant resonance peaks 1400 that start in close proximity to the 30 keV (3×10⁻² MeV) transition between epithermal and fast neutron energy regions. The resonance peaks continue at intervals as energies increase therefrom. The fast neutron scattering occurs either in the relatively lower energy region having discrete resonance peaks or the relatively higher energy, unresolved resonance region, where the resonance peaks are so crowded and close in energy they effectively no longer look like individual resonances. Fast-S materials selected for NBC 200 preferably have a resonance region that starts at the upper limit of the target energy (e.g., epithermal) region needed for a specific treatment. Magnesium has a first resonant peaks at 20 keV, then again at −80 keV and higher energies. Neutrons having fast energies coinciding with these resonant peaks are relatively more likely to be scattered and thus reduced in energy towards the epithermal region. Conversely, neutrons in the epithermal region are less likely to be scattered by these materials as they travel through, thus permitting the neutrons to remain within the desired energy region until exiting NBC 200 and reaching the treatment site in the patient.

Aluminum is a highly versatile material with structural strength, relative ease of manufacture, and the ability to be combined with other elements in a wide variety of compounds. Aluminum alloy blends are divided into three different categories based on the alloying material, which can be other elements such as magnesium and silicon with desirable scattering properties. Aluminum 6000 series alloys include greater than 97% aluminum (by weight percent) and the remaining elements can be either silicon or magnesium. Aluminum 5000 series alloys include greater than 90% aluminum with the dominant alloying element being up to 10% magnesium. Some 5000 series cast metals can have as little as 32% aluminum with the remainder being magnesium. Aluminum 4000 series alloys include greater than 85% aluminum and up to 12.5% silicon, with the remaining balance being magnesium, manganese, or copper. Some 4000 series cast metals can have up to 22% silicon.

The scattering properties of fluorine can be utilized effectively within NBC 200 with one or more other materials in the form of a compound. For example, fluorine can be combined as an alloy with aluminum (e.g., AlF₃), titanium (e.g., TiF₃), barium (e.g., BaF₂), bismuth (e.g., BiF₃), lead (e.g., PbF₂), tungsten (e.g., WF₆), vanadium (e.g., VF₃), magnesium (e.g., MgF₂), calcium (e.g., CaF₂), or with carbon and hydrogen (e.g., ethylene tetrafluoroethylene (ETFE, C₄H₄F₄)). By mass the fluorine content can generally range from approximately 15% (as with lead fluoride (PbF₂)) to 54% (as with TiF₃) or 68% (as with AlF₃). FIG. 14B is a graph depicting cross sections for magnesium fluoride (MgF₂), lead fluoride (PbF₂), and bismuth fluoride (BiF₃). The term fluorine as used herein is intended to cover the element itself and fluorides.

The materials within NBC 200 that undergo relatively high radiation exposure, such as within central region 210, preferably exhibit sufficient radiation resistance to prevent degradation within NBC 200. In various embodiments the materials of NBC 200 are radiation tolerant up to a minimum of 10,000 Gray (Gy). Some fluorine bearing polymers, like polytetrafluoroethylene (PTFE, C₂F₄), have poor radiation resistance, where physical changes (e.g., degradation, crumbling) can occur as low as 100 Gy, and are not suited for NBC 200.

The fast-scattering effect of aluminum and fluorine can be enhanced by combination of those elements with magnesium into compounds of two or more elements, such as magnesium fluoride (see FIG. 14A) and the aluminum 4000, 5000, and 6000 series. Magnesium can be combined with numerous other S materials desired for scattering, such as zinc, manganese, and silicon for use within NBC 200. All aforementioned Fast-S example materials can be used in the embodiments of NBC 200 alone, in combination with each other, or in combination with other materials (e.g., Epi-S, NR-S, Ab, R) to achieve the desired structural characteristics and scattering capability.

### Example Epi-S Materials

Epi-S elements for NBC 200 can have an atomic number (Z) of 12 or greater. Particularly useful examples of these Epi-S elements are titanium and vanadium, and to a lesser extent magnesium. FIG. 14C is a graph depicting cross sections for titanium, vanadium, and magnesium in the epithermal energy region, from which the large resonant cross-sections 1402 for titanium and vanadium in the upper epithermal range is visible. Titanium and vanadium can be used in combination with each other, in combination with other elements, or alone within NBC 200. Titanium and vanadium can be readily combined with aluminum (or another Fast-S material) to take advantage of its Fast-S scattering potential. Common titanium alloys have both vanadium and aluminum, and the aluminum content can range from 6 to 30% (by weight percent) while the vanadium content can range from 2.5 to 4.0%. Alloys of titanium and aluminum (without vanadium) can be used and can have as little as 12% or as much as 35% aluminum. Alloys of titanium and vanadium (without aluminum) can be used and can have as little as 10% vanadium up to 81% vanadium, with the remainder being titanium. Like aluminum, some titanium compounds can contain as much as 10% silicon, while vanadium can be a silicide (VS₂, VS₃, etc.). Other examples of Epi-S materials include scandium (Sc), nickel (Ni), and zinc (Zn). All aforementioned Epi-S example materials can be used in the embodiments of NBC 200 alone, in combination with each other, or in combination with other materials (e.g., Fast-S, NR-S, Ab, R) to achieve the desired structural characteristics and scattering capability.

### Example NR-S Materials

Certain elements exhibit a broad non-resonant region for cross-sections at lower energies after termination of the 1/V region and extending to higher energies where resonance cross-sections commence. The non-resonance region of the cross-section can have a constant slope, e.g., flat, and can be decreasing (to a substantially lesser extent than in the 1/V region) or constant. These elements are referred to herein as non-resonant scattering materials (“NR-S”), examples of which include hydrogen, lithium, boron, beryllium, carbon, nitrogen, and oxygen. In many embodiments, the NR-S material exhibits deviation of 20% or less over the target energy range (e.g., epithermal), more preferably 10% or less (e.g., like carbon's cross-section up to ˜120 keV).

FIG. 14D is a graph depicting cross-sections for carbon, beryllium, and water (H₂O). Carbon and beryllium have a non-resonant scattering region 1404 with a slope at or near zero from approximately 0.05 eV to 50 keV. Water has a non-resonant scattering region with a relatively small negative slope from approximately 0.05 eV to 10 keV. All NR-S elements can be used in the embodiments of NBC 200 alone, in combination with each other, or in combination with other materials (e.g., Fast-S, Epi-S, R) to achieve the desired structural characteristics and scattering capability.

### Example Ab Materials

Certain elements exhibit a broad 1/V region with significant cross-sections that can extend across the thermal energy region and at least a substantial portion of the epithermal energy region. The 1/V region of the cross-sections can have a constant slope, e.g., flat, and can be decreasing with a substantial slope. These elements are particularly useful at absorbing neutrons and are referred to herein as absorption materials (“Ab”), examples of which include hydrogen, lithium, boron, and carbon. These materials can be referred to either as Ab materials or as NR-S materials given the context. FIG. 14E is a graph depicting cross-sections for carbon, boron, and lithium. Boron has a 1/V region 1410 that extends across the thermal energies and most of the epithermal energy region up to approximately 10 keV, while lithium's 1/V region 1410 extends across both the thermal and epithermal regions up to approximately 50 keV.

FIG. 14O depicts lithium titanate, alongside two Epi-S materials, a titanium-aluminum-vanadium alloy (Ti-6Al-4V) and titanium dioxide (TiO₂). FIG. 14P depicts titanium diboride, alongside the Epi-S material Ti6Al4V and a Fast-S aluminum-magnesium compound. FIG. 14Q depicts boron carbide (B₄C) alongside the Epi-S and NR-S material titanium aluminate and the Fast-S aluminum-magnesium compound. As can be seen here lithium titanate, titanium diboride, and boron carbide have significant 1/V regions that extend across most of the epithermal energy range. Lithium titanate and titanium diboride both have significant resonance peaks at the upper epithermal energy range, and boron carbide has a significant cross section across the entire epithermal energy range, which contributes to the usefulness of these materials in scattering epithermal neutrons for absorption. As such, materials having lithium or boron make excellent neutron absorbers and can also be used as significant Epi-S materials.

Ab materials can be used in regions of NBC 200 where elimination of the neutrons is desirable, such as near the rear and lateral extremes of NBC 200, and near the front on surfaces surrounding output 202. All Ab elements can be used in the embodiments of NBC 200 alone, in combination with each other, or in combination with other materials (e.g., Fast-S, Epi-S, NR-S, R) to achieve the desired structural characteristics and absorptive capability. Additional examples of combinations include hydrogenous materials such as polymers like polyethylene (PE) and boronated polyethylene (B-PE). Additional examples of Ab materials include cadmium (Cd), gadolinium (Gd), indium (In), and hafnium (Hf)

### Example R Materials

Certain elements tend to redirect neutrons through elastic scattering within minimal neutron energy loss. This process is proportional to the Z value of the element in contrast to the structure of the cross-section. Each element with a Z value of from and including 74 (tungsten) up to and including 92 (uranium) are elements that are particularly adept at redirecting neutrons, and are referred to generally herein as redirection (“R”) materials. These materials are also adept at absorbing gamma radiation. FIG. 14F is a graph depicting cross-sections for R materials tungsten, bismuth, and lead across thermal and epithermal energies, while FIG. 14G is a graph depicting the same cross-sections for energies from one keV to one MeV. Of the three materials tungsten has the largest cross-section outside of resonance peaks and thus the greatest scattering capability. All R elements can be used in the embodiments of NBC 200 alone, in combination with each other (e.g., lead and bismuth alloys (e.g., 40-50% Pb by weight)), or in combination with other materials (e.g., Fast-S, Epi-S, NR-S, Ab) in regions where increased redirection is desired and/or increased gamma radiation shielding. For example, NBC 200 can use one or more R materials in combination with one or more S materials (e.g., PbF₂ and BiF₃ as depicted in FIG. 14B) in order to redirect neutrons with minimal energy loss into collisions with materials (Fast-S, Epi-S, NR-S) having the desired scattering characteristics to locally intensify the number of energy reducing neutron collisions that take place (referred to generally herein as an “R+S” combination).

### Example Carbide, Nitride, Oxide Compounds for Non-Resonant Effects

Carbon, nitrogen and oxygen are NR-S materials that can be combined with other scattering elements to add or enhance non-resonant scattering capabilities of the overall material. For example, aluminum is a Fast-S element that can be formed as an oxide (e.g., Al₂O₃), carbide (e.g., Al₄C₃), and nitride (e.g., AlN). Examples of each are depicted in the cross-section versus energy graph of FIG. 14H. When aluminum is formed into an oxide, carbide, or nitride compound the resulting material's resonant cross-sections are equal to that of bare aluminum, but the cross-sections within gaps are significantly higher, as evidenced by the cross-sections within gap 1420 in FIG. 14H. As such, aluminum has a higher average cross-section in the epithermal and fast energy regions resulting from the addition of the NR-S material. Similar gap-raising results can be obtained when other Fast-S, Epi-S, and NR-S elements (e.g., magnesium, titanium, vanadium, beryllium, lithium, boron, hydrogen and/or fluorine) are formed into oxides, nitrides, carbides, or carbonates. Aluminum oxides and titanium oxides can be combined with other elements (X) to become aluminates (X+Al₂O₃) or titanates (X+TiO₂). When such combinations are formed, the oxygen, nitrogen, or carbon content is preferably 20-80% by mass of the overall material. Oxides, nitrides, and carbides are advantageous in that they can be readily sintered into numerous complex shapes. Any of the embodiments of NBC 200 can be implemented with one or more oxide, carbide, and/or nitrides.

### Example Embodiments of Central Regions

Selection of materials and material dimensions is dependent on the energy profile of neutrons generated by a particular target 60. Because the neutron energy profiles are dependent on the design, material, and construction of target 60 as well as the characteristics (e.g., energy and current) of the incident charged particle beam, or other mechanism utilized for generating the neutrons, numerous examples of embodiments of NBC 200 are disclosed herein.

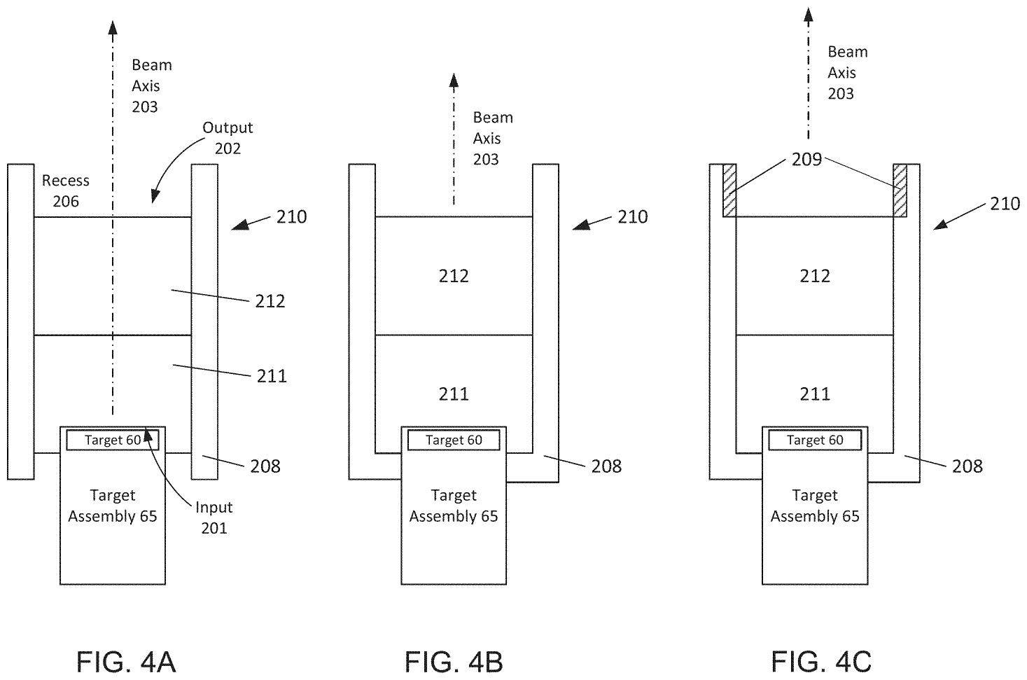

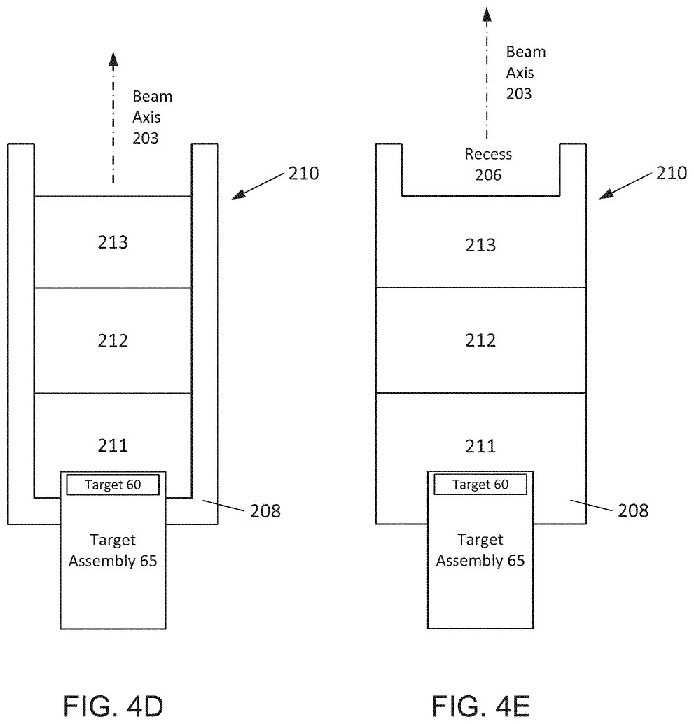

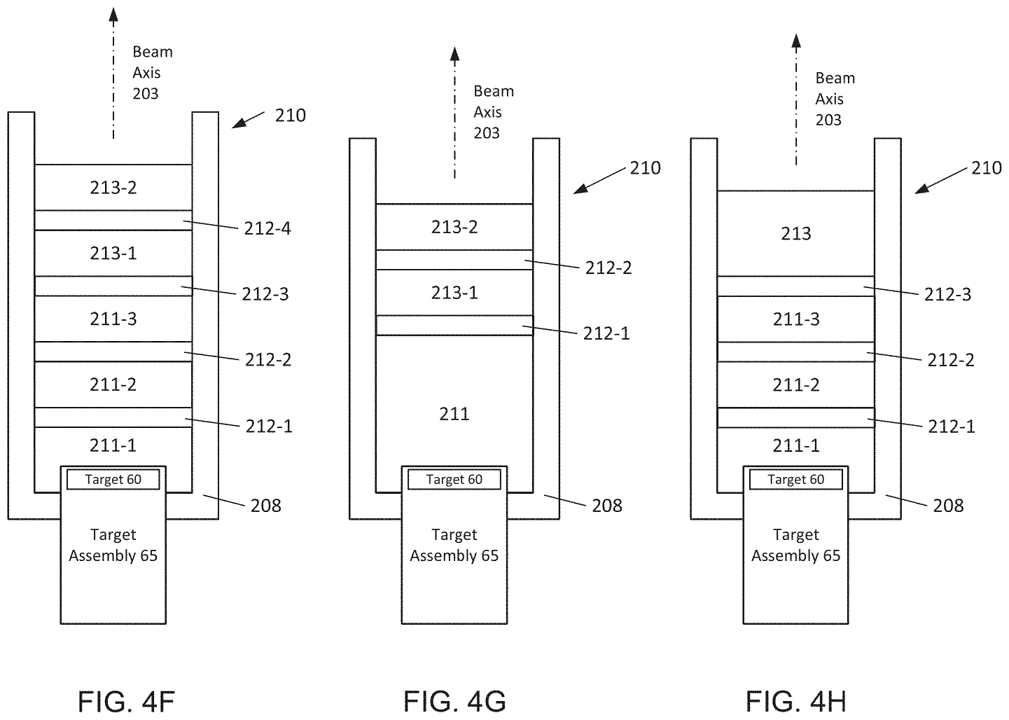

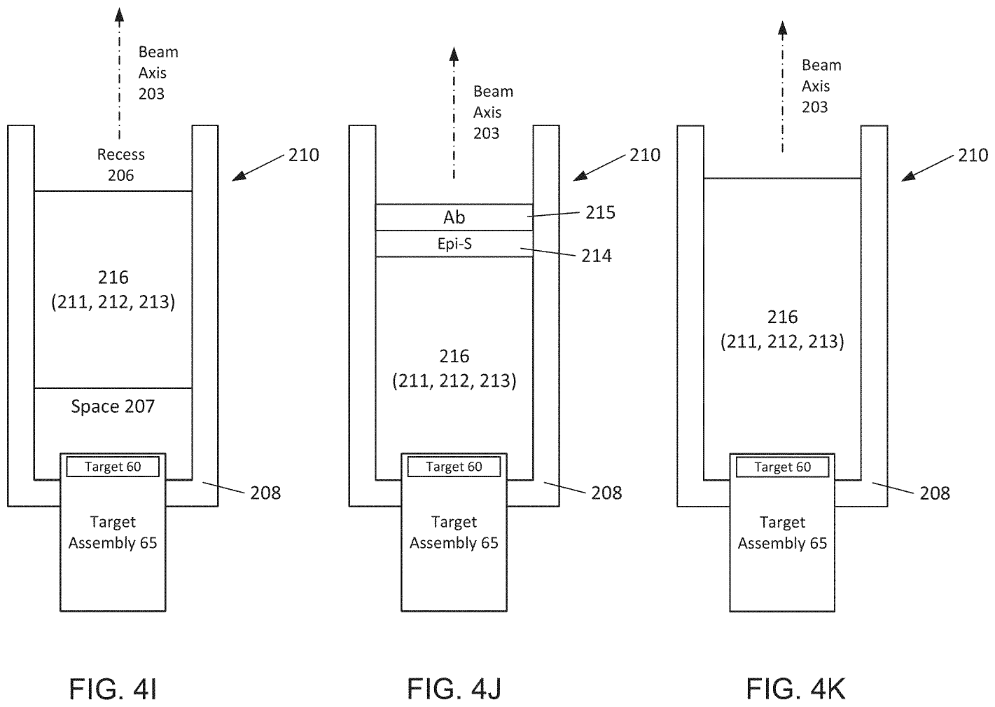

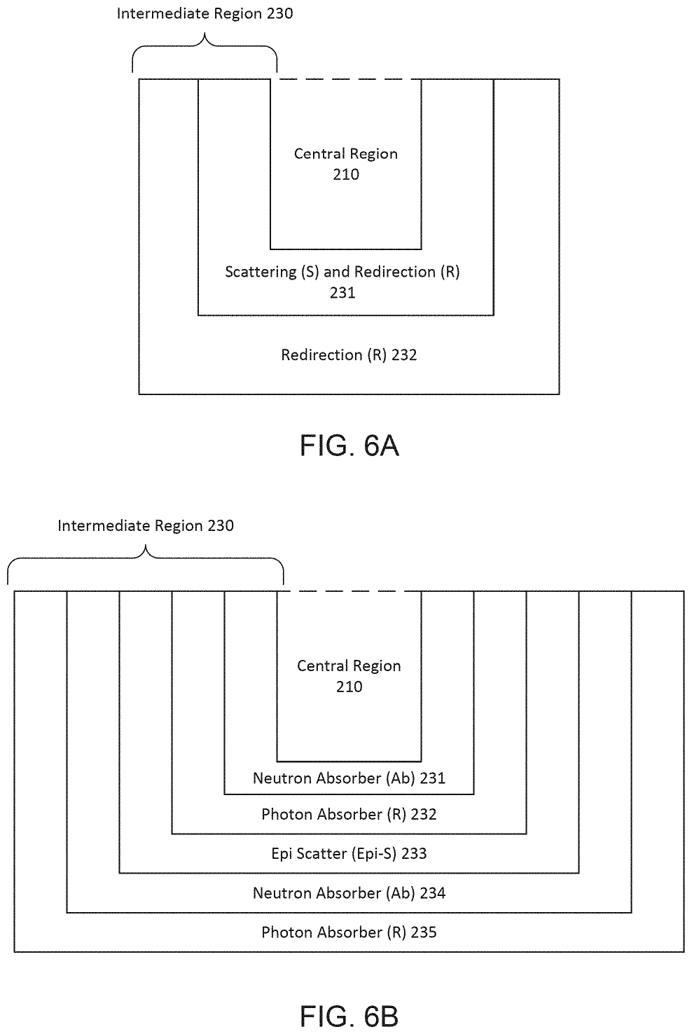

Some of these embodiments utilize stacked or layered arrangements where sections with different materials are placed adjacent to each other to form a sequence that acts upon neutrons passing therethrough. Such stacked arrangements permit more complex functions to be performed on the neutrons in terms of the scattering of different energies, redirection, and absorption. Stacked layers permit a relatively high degree of tunability of effects for a particular raw neutron profile. These effects can be tuned axially and laterally along all directions of neutron propagation from target 60 by stacking layers along axis 203 (see FIGS. 4A-4E and 4G-4K) and laterally along directions 204, for example, as in the sectional arrangement of central region 210 and intermediate region 230 (see FIGS. 6A-6B).

FIGS. 4A-4K are axial cross-sectional views of example embodiments of central region 210 having different arrangements of one or more materials therein. In these embodiments central region 210 is bisected with beam input 201 oriented at bottom and beam output 202 at top. Each of the embodiments includes a shell 208 that extends from input 201 to output 202. Shell 208 can be in the form of a tubular wall, e.g., with a circular, elliptical, polygonal, or other lateral cross-sectional profile. These embodiments can be implemented without shell 208 as well (e.g., FIG. 4E). Surrounded by shell 208 are one or more sections or regions (e.g., 211, 212, and/or 213) where each is traversed by beam axis 203. Each section can contain a different material and have different dimensions from all other sections (i.e., such that no two sections are the same), or the same or similar sections can be repeated (in terms of dimensions and/or materials), and combined with one or more sections having a different material and/or dimension. Central region 210 can include one, two, three, four or more distinct sections each of which may be repeated two or more times.

In these embodiments, shell 208 can extend upstream past the installed position of target 60. Shell 208 can terminate upstream within NBC 200 or can extend all the way to the upstream terminus of NBS 200 (e.g., the rear-most face). The upstream terminus of shell 208 can be offset apart from assembly 65 as shown in FIG. 4A, or can include an inwardly extending flange or lip that closes in on assembly 65 as shown in FIG. 4B. Shell 208 can form a relatively low resistance pathway for neutron travel as compared to pathways adjacent to shell 208. For example, shell 208 can be an S material with relatively low redirection propensity, such that neutrons entering shell 208 are transported to beam output 202 with any requisite down scattering to the epithermal range.

In the embodiments of FIGS. 4A-4C, a first section 211 is adjacent beam input 201 and a second section 212 is present between section 211 and beam output 202 such that sections 211 and 212 are stacked. Shell 208 can be supplemented with one or more other materials at output 202 as depicted in FIG. 4C by output liner 209, which can be an S and/or an R material to aid in forward-directed beam formation. FIG. 4D depicts an example embodiment with three stacked sections 211-213. Neutrons passing through central region 210 from target 60 will pass through sections 211, 212, and 213 sequentially. FIG. 4E depicts an example embodiment similar to FIG. 4D but without a shell 208. Recess 206 is present within outwardly extending sidewalls of section 213.

FIGS. 4F-4H depict example embodiments where at least one of sections 211, 212, and 213 is repeated in an interlaced fashion with at least one of the other sections 211, 212, and 213. Neutrons passing through central region 210 from target 60 will pass through sections 211, 212, and 213 in the order of their placement from upstream to downstream along axis 203. In FIG. 4F, sections 211-1, 211-2, and 211-3 are separated by one of sections 212-1 and 212-2. Sections 211-3 and 213-1 are separated by section 212-3. Sections 213-1 and 213-2 are separated by section 212-4. In FIG. 4G, section 211 includes a combination of an R material with a first type of S material (S1) without any additional stacking or layering within section 211. In FIG. 4G, a section 212-1 is located axially downstream of section 211, followed by a section 213-1, followed by a section 212-2, followed by a section 213-2. In FIG. 4H, sections 211-1 through 211-3 and 212-1 through 212-3 are configured similarly to the embodiment of FIG. 4F, with just a single section 213 located downstream. Other stacked configurations can be utilized for central region 210 of which these are but a few examples.

In the embodiment of FIG. 4I, an interior non-solid space 207 is present at beam input 201, such that neutrons emitted by target 60 cross a non-solid space before reaching contiguous solid or semi-solid section 216, which can be a single section containing a mixture of one or more of the materials used in discrete sections 211, 212, and 213 of the aforementioned embodiments. Put differently, one, two or three of the materials of discrete sections 211, 212, and 213 can be intermixed to form a single section 216. In FIG. 4J, section 216 is followed by axially downstream by section 214 which contains an epithermal scattering material (Epi-S) followed by section 215 containing an absorption material (A). Such a configuration can be utilized, for example, to remove lower energy epithermal neutrons from the resulting beam 70 by down scattering neutrons with section 212 to an energy range more readily absorbed by section 213. FIG. 4K is an example of central region 210 with shell 208 and only one section 216.

Each section 211, 212, and 213 preferably includes or is formed of a different scattering (S) material, such that the S material of section 211 (indicated by S1) is different from the S material of section 212 (indicated by S2), which in turn is different from the S material of section 213 (indicated by S3 for embodiments having section 213). Shell 208, if present, can also include an S material that is the same (S1, S2, or S3) or different (S4) than the S materials of sections 211, 212, and 213. The S material for sections 211, 212, 213 and shell 208 can be Fast-S, Epi-S, or NR-S types, or a combination of any two or three types. In embodiments where the desired energy output is in the epithermal range, the S materials are preferably either Fast-S or NR-S materials.

The following tables set forth example embodiments of the material combinations that can be used within central region 210. Each of sections 211, 212, and 213 can be combined with a redirection (R) material as desired. Combination with an R material intensifies the amount of local scattering, as neutrons passing through can be redirected numerous times by the R material without significant loss of energy, and thus increase the likelihood of encountering the S material and scattering in the preferential manner according to that S materials resonance or non-resonance cross-section.

Table 1 sets forth four examples for embodiments having two sections 211 and 212 and no shell 208. Tables 2A and 2B set forth sixteen examples for embodiments having two sections 211 and 212 and a shell 208, which can be of the same material (S1 or S2) as sections 211 and 212 or a different material (S3), with or without an R material. To illustrate by reference to Table 1, example 1 (“Ex. 1”) sets forth a configuration where section 211 includes a combination of an R material and an S1 material and section 212 includes an S2 material with no R material (as indicated by the absence of “R”). Example 2 (“Ex. 2”) sets forth a configuration where section 211 includes an S1 material with no R material and section 212 includes a combination of an R material and an S2 material. Example 3 (“Ex. 3”) sets forth a configuration where section 211 includes an S1 material and section 212 includes an S2 material neither having an R material. Example 4 (“Ex. 4”) sets forth a configuration where section 211 includes a combination of an R material and an S1 material and section 212 includes a combination of an R material and an S2 material, where the R materials of example 4 can be the same or different. The same convention is used for Tables 2A and 2B.

TABLE 1

| | | | | |
|---|---|---|---|---|
| Section | Ex. 1 | Ex. 2 | Ex. 3 | Ex. 4 |

| | | | | | | | |
|---|---|---|---|---|---|---|---|
| 211 | R | S1 | | S1 | S1 | R | S1 |
| 212 | | S2 | R | S2 | S2 | R | S2 |

TABLE 2A

| Section | Ex. 1 | | Ex. 2 | | Ex. 3 | Ex. 4 | | Ex. 5 | | Ex. 6 | | Ex. 7 | | Ex. 8 |
|---|---|---|---|---|---|---|---|---|---|---|---|---|---|---|
| 211 | R | S1 | | S1 | S1 | R | R | R | S1 | | S1 | R | S1 | S1 |
| 212 | | S2 | R | S2 | S2 | R | R | | S2 | R | S2 | R | S2 | S2 |
| 208 | | S1 or | | S1 or | S1 or | | S1 or | | S3 | | S3 | | S3 | S3 |
| | | S2 | | S2 | S2 | | S2 | | | | | | | |

TABLE 2B

| Section | Ex. 9 | | Ex. 10 | | Ex. 11 | | Ex. 12 | | Ex. 13 | | Ex. 14 | | Ex. 15 | | Ex. 16 | |
|---|---|---|---|---|---|---|---|---|---|---|---|---|---|---|---|---|
| 211 | R | S1 | | S1 | | S1 | R | R | R | S1 | | S1 | R | S1 | | S1 |
| 212 | | S2 | R | S2 | | S2 | R | R | | S2 | R | S2 | R | S2 | | S2 |
| 208 | R | S1 or | R | S1 or | R | S1 or | R | S1 or | R | S3 | R | S3 | R | S3 | R | S3 |
| | | S2 | | S2 | | S2 | | S2 | | | | | | | | |

The examples set forth in the remaining Tables 3, 4A, 4B, 4C, and 4D follow the same convention as Tables 1, 2A, and 2B. Table 3 sets forth eight examples for embodiments of central region 210 having three sections 211, 212 and 213 and no shell 208, whereas Tables 4A, 4B, 4C, and 4D set forth 32 examples for embodiments having three sections 211, 212, and 213 and a shell 208. In these embodiments shell 208 can be of the same material as one of sections 211, 212, and 213 (S1, S2, or S3) or a different material (S4), with or without an R material. If the same material is used, then shell 208 can be contiguous (e.g., seamless) with and unified with the section (211, 212, or 213) having the same material. If shell 208 is unified with section 212 for example, then shell 208 can enclose or encapsulate all or substantially all of section 211 (or any combination of sections contained within shell structure 208), with beam axis 203 extending parallel to the walls of section 208 and then traversing section 208/212. Similarly, if shell 208 is unified with section 213, then it can enclose or encapsulate sections 211 and 212.

TABLE 3

| Section | Ex. 1 | | Ex. 2 | | Ex. 3 | | Ex. 4 | | Ex. 5 | | Ex. 6 | | Ex. 7 | | Ex. 8 |
|---|---|---|---|---|---|---|---|---|---|---|---|---|---|---|---|
| 211 | R | S1 | | S1 | | S1 | R | S1 | R | S1 | | S1 | R | S1 | S1 |
| 212 | | S2 | R | S2 | | S2 | R | S2 | | S2 | R | S2 | R | S2 | S2 |
| 213 | | S3 | | S3 | R | S3 | | S3 | R | S3 | R | S3 | R | S3 | S3 |

TABLE 4A

| Section | Ex. 1 | | Ex. 2 | | Ex. 3 | | Ex. 4 | | Ex. 5 | | Ex. 6 | | Ex. 7 | | Ex. 8 |
|---|---|---|---|---|---|---|---|---|---|---|---|---|---|---|---|
| 211 | R | S1 | | S1 | | S1 | R | S1 | R | S1 | | S1 | R | S1 | S1 |
| 212 | | S2 | R | S2 | | S2 | R | S2 | | S2 | R | S2 | R | S2 | S2 |
| 213 | | S3 | | S3 | R | S3 | | S3 | R | S3 | R | S3 | R | S3 | S3 |
| 208 | | S1, | | S1, | | S1, | | S1, | | S1, | | S1, | | S1, | S1, |
| | | S2, | | S2, | | S2, | | S2, | | S2, | | S2, | | S2, | S2, |
| | | or S3 | | or S3 | | or S3 | | or S3 | | or S3 | | or S3 | | or S3 | or S3 |

TABLE 4B

| Section | Ex. 9 | | Ex. 10 | | Ex. 11 | | Ex. 12 | | Ex. 13 | | Ex. 14 | | Ex. 15 | | Ex. 16 | |
|---|---|---|---|---|---|---|---|---|---|---|---|---|---|---|---|---|
| 211 | R | S1 | | S1 | | S1 | R | S1 | R | S1 | | S1 | R | S1 | | S1 |
| 212 | | S2 | R | S2 | | S2 | R | S2 | | S2 | R | S2 | R | S2 | | S2 |
| 213 | | S3 | | S3 | R | S3 | | S3 | R | S3 | R | S3 | R | S3 | | S3 |
| 208 | R | S1, | R | S1, | R | S1, | R | S1, | R | S1, | R | S1, | R | S1, | R | S1, |
| | | S2, | | S2, | | S2, | | S2, | | S2, | | S2, | | S2, | | S2, |
| | | or S3 | | or S3 | | or S3 | | or S3 | | or S3 | | or S3 | | or S3 | | or S3 |

TABLE 4C

| Section | Ex. 17 | | Ex. 18 | | Ex. 19 | | Ex. 20 | | Ex. 21 | | Ex. 22 | | Ex. 23 | | Ex. 24 |
|---|---|---|---|---|---|---|---|---|---|---|---|---|---|---|---|
| 211 | R | S1 | | S1 | | S1 | R | S1 | R | S1 | | S1 | R | S1 | S1 |
| 212 | | S2 | R | S2 | | S2 | R | S2 | | S2 | R | S2 | R | S2 | S2 |
| 213 | | S3 | | S3 | R | S3 | | S3 | R | S3 | R | S3 | R | S3 | S3 |
| 208 | | S4 | | S4 | | S4 | | S4 | | S4 | | S4 | | S4 | S4 |

TABLE 4D

| Section | Ex. 25 | | Ex. 26 | | Ex. 27 | | Ex. 28 | | Ex. 29 | | Ex. 30 | | Ex. 31 | | Ex. 32 | |
|---|---|---|---|---|---|---|---|---|---|---|---|---|---|---|---|---|
| 211 | R | S1 | | S1 | | S1 | R | S1 | R | S1 | | S1 | R | S1 | | S1 |
| 212 | | S2 | R | S2 | | S2 | R | S2 | | S2 | R | S2 | R | S2 | | S2 |
| 213 | | S3 | | S3 | R | S3 | | S3 | R | S3 | R | S3 | R | S3 | | S3 |
| 208 | R | S4 | R | S4 | R | S4 | R | S4 | R | S4 | R | S4 | R | S4 | R | S4 |

The S materials (e.g., S1 through S4) can include one or more that exhibit resonances within the energy range of neutrons produced by the target 60 and above the desired output energy range, for example, a Fast-S material when the desired energy range is epithermal. The S materials can also include one or more NR-S materials that exhibit non-resonance characteristics within the energy range of neutrons produced by the target 60 and at least above the desired output energy range, for example, to fill gaps between Fast-S material resonances. In some example embodiments, central region 210 includes a combination of at least one S material exhibiting resonance characteristics, with at least one S material exhibiting non-resonance characteristics. These materials can be separated in different sections or combined in a single section (e.g., aluminum oxide). Table 5 sets forth eight examples of configurations of the S materials for embodiments having two sections 211 (S1) and 212 (S2). Tables 6A and 6B sets forth fourteen example configurations of the S materials for embodiments having three sections 211 (S1), 212 (S2) and 213 (S3). For example, referring to Ex. 1 of Table 5, the S1 material includes both a Fast-S material (indicated by the X in the F column) and an NR-S material (indicated by the X in the NR column), while the S2 material includes only a Fast-S material (indicated by the X in the F column and the absence of an X in the NR column). The remaining examples use the same convention in Tables 5, 6A, and 6B. The example configurations of Table 5 can be used with all two section examples of Tables 1, 2A, and 2B, while the example configurations of Tables 6A and 6B can be used with all three section examples of Tables 3, 4A, 4B, 4C, and 4D.

TABLE 5

| | Ex. 1 | | Ex. 2 | | Ex. 3 | | Ex. 4 | | Ex. 5 | | Ex. 6 | | Ex. 7 | | Ex. 8 | |
|---|---|---|---|---|---|---|---|---|---|---|---|---|---|---|---|---|
| S Mat. | F | NR | F | NR | F | NR | F | NR | F | NR | F | NR | F | NR | F | NR |

| | | | | | | | | | | | | | | | |
|---|---|---|---|---|---|---|---|---|---|---|---|---|---|---|---|
| S1 (211) | X | X | X | | X | | X | X | | X | X | X | X | | X |
| S2 (212) | X | | X | X | X | X | | | X | X | X | | X | X | X |

TABLE 6A

| | Ex. 1 | | Ex. 2 | | Ex. 3 | | Ex. 4 | | Ex. 5 | | Ex. 6 | | Ex. 7 | |
|---|---|---|---|---|---|---|---|---|---|---|---|---|---|---|
| S. Mat | F | NR | F | NR | F | NR | F | NR | F | NR | F | NR | F | NR |
| S1 (211) | X | X | X | | X | | | X | X | | X | | X | X |
| S2 (212) | X | | X | X | X | | X | | | X | X | | | X |
| S3 (213) | X | | X | | X | X | X | | X | | | X | X | |

TABLE 6B

| | Ex. 8 | | Ex. 9 | | Ex. 10 | | Ex. 11 | | Ex. 12 | | Ex. 13 | | Ex. 14 | |
|---|---|---|---|---|---|---|---|---|---|---|---|---|---|---|
| S. Mat | F | NR | F | NR | F | NR | F | NR | F | NR | F | NR | F | NR |
| S1 (211) | X | X | X | X | X | | X | X | X | X | X | | X | X |
| S2 (212) | X | | X | X | X | X | X | | X | X | | X | | X |
| S3 (213) | | X | X | | X | X | X | X | X | X | | X | | X |

FIGS. 14I-14N are cross-section v. energy graphs that illustrate the overall effects of combining multiple materials having different cross-sectional characteristics, and are just a few of the many combinations of materials that can be used for sections 211, 212, and 213. FIG. 14I depicts three materials: carbon (NR-S material), magnesium fluoride (Fast-S material), and aluminum (Fast-S material) that can be used as S1, S2, and S3 in any order. FIG. 14J depicts three materials: carbon (NR-S material), bismuth fluoride (Fast-S and R material), and aluminum oxide (Fast-S and NR-S material) that can be used as S1, S2, and S3 in any order. FIG. 14K depicts three materials: beryllium oxide (NR-S material), lead fluoride (Fast-S and R material), and aluminum (Fast-S material) that can be used as S1, S2, and S3 in any order. FIG. 14L depicts three materials: beryllium oxide (NR-S material), magnesium fluoride (Fast-S material), and aluminum (Fast-S material) that can be used as S1, S2, and S3 in any order. FIG. 14M depicts three materials: ETFE (Fast-S and NR-S material), lead fluoride (Fast-S and R material), and aluminum (Fast-S material) that can be used as S1, S2, and S3 in any order. FIG. 14N depicts three materials: ETFE (Fast-S and NR-S material), bismuth fluoride (Fast-S and R material), and aluminum oxide (Fast-S and NR-S material) that can be used as S1, S2, and S3 in any order. As can be seen from the graphs the combination of Fast-S and NR-S materials (such as beryllium or beryllium oxide) provides more uniform cross-sections across a wide energy range of fast and epithermal energies.

In some embodiments upstream section 211 can include an S1 material with scattering resonance cross-sections at relatively high energies (e.g., fast) within the range of neutron energies produced by target 60, with the immediately downstream section 212 being an S2 material with one or more resonance cross-sections at relatively lower energies, and the most downstream section 213 being an S3 material with one or more resonance cross-sections at still lower energies at or just above the desired energy range (e.g., epithermal) of the converted beam 70. Additionally, or alternatively, the sections 211, 212, and 213 can have resonance cross sections at the same or similar energies, but with upstream section 211 having the relatively largest cross-section at relatively high energies (e.g., fast) within the range of neutron energies produced by target 60 as compared to the cross-sections of sections 212 and 213 at the same or similarly high energies, immediately downstream section 212 having the relatively largest cross-section at a relatively lower energies as compared to the cross-sections of sections 211 and 213 at the same or similar energies, and the most downstream section 213 having the relatively largest resonance cross-section at still lower energies at or just above the desired energy range (e.g., epithermal) of the converted beam 70.

The energies of these neutrons can be progressively lowered by each section such that the neutrons exiting output 202 have been down-scattered into the desired range. Similarly, in some embodiments the materials used in the various sections are selected to have resonance energies that do not fully overlap and at least partially complement each other to provides more uniform scattering. For example magnesium fluoride and aluminum have resonances that only partially overlap from approximately 20 keV to 70 keV as shown in FIGS. 14A, 14I, and 14L.

The R material can also provide benefits such as gamma radiation absorption. Placement of the R material in the relatively more upstream section (e.g., 211) allows more time and volume for other parts of NBC 200 to redirect neutrons that are inadvertently redirected out of section 211, as these neutrons can be redirected back into central region 210 by intermediate region 230. The downstream section(s) (e.g., 212, 213) can perform scattering without substantial redirection to maintain the forward-directed beam shape.

As a combination of an R and S material, lead fluoride has diverse functions due to the nuclear properties of lead and fluorine, as well as several material properties. The fluorine atoms have a series of large resonant scattering peaks, amplified due to the high mass density of the compound, that preferentially scatter neutrons above the epithermal range without excessive energy loss (9.5% on average). The lead can serve two roles: as a reflector, only making small changes in the neutron energy spectrum until another fluorine atom is encountered, and a photon shield to attenuate prompt gamma rays generated within the NBC 200. This material will lead to low neutron flux levels at fluorine peak energies and higher fluxes at valley energies. Energy reduction of the valley fluxes can be accomplished by alternating sections of lead fluoride with a non-resonance material like beryllium or beryllium oxide. Thicknesses of sections of beryllium can be set corresponding to the amount of material that on average will provide the appropriate number of collisions to advance neutrons from one fluorine resonance to the next. As shown in the equations above beryllium is much more efficient at this process than lead or fluorine, thus, the use of beryllium or beryllium oxide can reduce the overall size of central region 210.

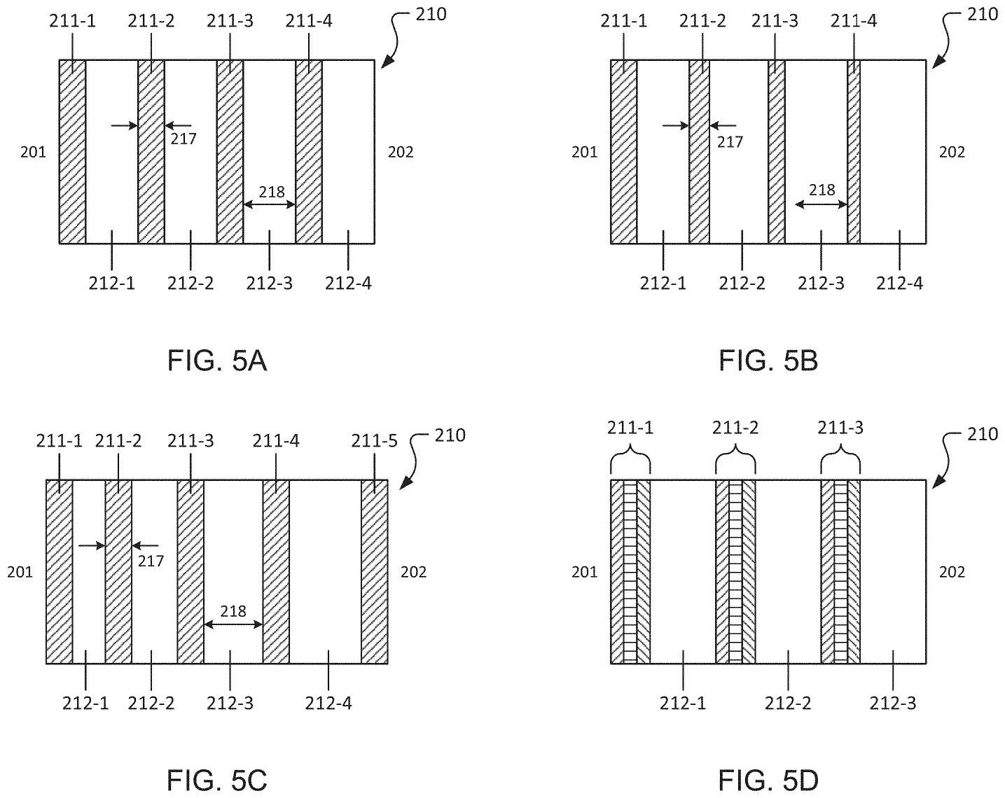

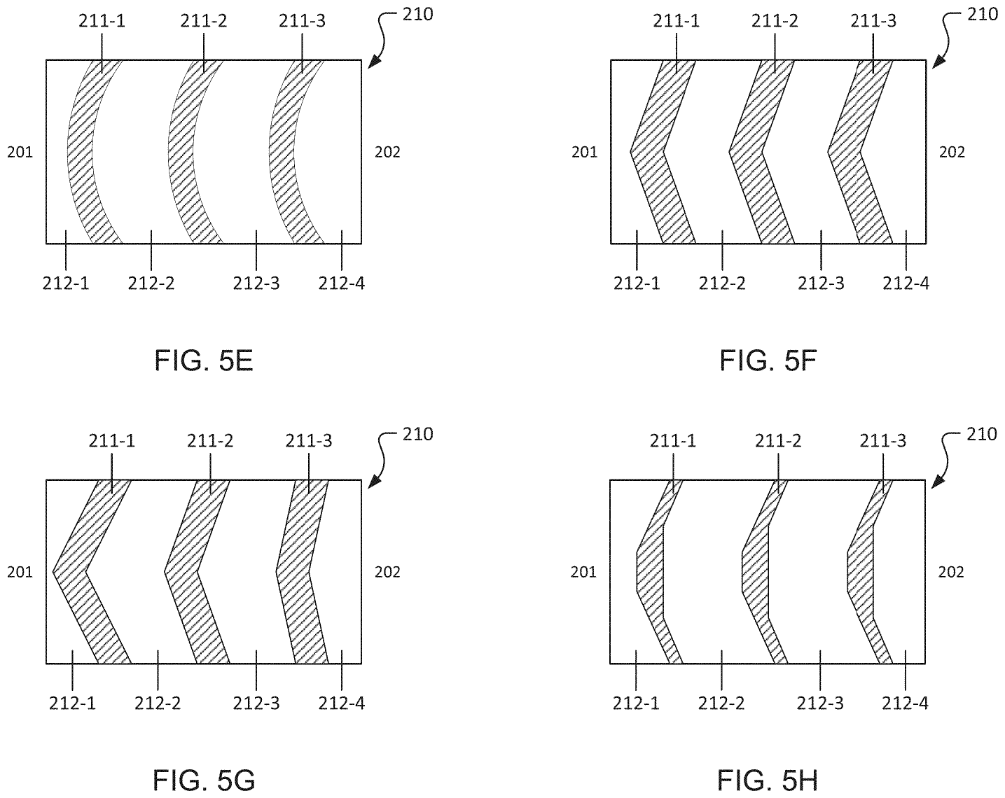

The stacked sections within central region 210 can be configured in numerous ways, with different shapes, thicknesses, and combinations as shown in the example embodiments depicted in the cross-sectional views of FIGS. 5A-5H. In these embodiments central region 210 is bisected along beam axis 203, with beam input 201 at left and beam output 202 at right. Each central region 210 has sections 211 interleaved with sections 212, where sections 211 vary in relative shape, dimension, and/or spacing with respect to sections 211 of the same embodiment (e.g., FIGS. 5B, 5C, 5G) or a different embodiment (e.g., FIGS. 5A, 5D, 5E, 5F, 5H). Emphasis of FIGS. 5A-5H is on the various cross-sectional shapes and relative spacing of layered sections, as opposed to variance of materials and functions within each group of sections (211, 212) and across different groups of sections (211, 212), of which numerous combinations can be utilized.

In the embodiment of FIG. 5A, each of sections 211 have the same (or substantially the same) thickness (indicated by arrows 217) and each of sections 212 have the same thickness (indicated by arrows 218). The thicknesses of the sections can be selected according to the needs of the particular beam conversion. For example, a thicker section having the function of scattering at a particular resonance can accomplish relatively more scattering with increasing thickness. In the embodiment of FIG. 5B, the thicknesses vary between sections 211 and also between sections 212. Here, the thicknesses 217 of sections 211 progressively decrease from input 201 to output 202. The thicknesses 218 of sections 212 can remain constant or progressively increase (as shown here). For example in FIG. 5B, the effect produced by sections 211 is progressively reduced from input 201 to output 202, while the effect produced by sections 212 is progressively increased. FIG. 5C depicts an embodiment where thickness 17 of sections 211 remains constant and thickness 218 of sections 212 progressively increases from input 201 to output 202.

FIG. 5D depicts an example embodiment where sections 211-1, 211-2, and 211-3 are each a grouping of multiple relatively thin materials. For example, each of sections 211 can include two, three (as shown here), four, or more of the same or different materials in thin sheets or plates placed in contact. Each sheet can be a different material from the other sheets of that section 211, or the sheets can alternate between materials. In some embodiments the sheets are the same material arranged in this fashion to facilitate construction of NBC 200, by allowing different sections 211 to be fabricated with different thicknesses determined by the sheet count.

FIG. 5E depicts an example embodiment where sections 211 have a curvilinear shape in cross-section, which can be bowl-shaped in three dimensions. The convex side of the bowl can face input 201 (as shown here) or the concave side of the bowl can face input 201 (i.e., reversed). FIG. 5F depicts an example where sections 211 have a V shape or a chevron shape in cross-section, which can be conical when viewed in three dimensions. The relatively more pointed side can face input 201 (as shown here) or the concave side can face input 201 (i.e., reversed). The depth (axially) of each section can be varied as shown in the example of FIG. 5G. Here, section 211-1 has a relatively deep shape that progressively shallows with section 211-3 and 211-3 (e.g., interior angle increases from section 211-1 to section 211-3). As with the other embodiments this configuration can be reversed. FIG. 5H depicts an example where each section 211 has a variable radial thickness such that the thickness dimension is non-constant when viewed laterally in cross-section. Variable radial thicknesses can be applied in all of the embodiments shown or contemplated herein. A variable thickness, such as a thicker region located centrally along the beam axis as with sections 211, can provide greater effect to the function of the material to neutrons passing through that region. The variable thickness can be reversed with thicker regions located off center as with sections 212.

These embodiments are illustrative of the many different configurations that can be implemented, and features of each embodiment can be combined with features of any and all of the other embodiments.

### Example Embodiments of Intermediate Regions

FIGS. 6A-6B are cross-sectional views depicting example embodiments of intermediate region 230. In FIG. 6A, region 230 includes a first section 231 adjacent to and in contact with the lateral and upstream sides of central region 210. Region 230 also includes a second section 232 adjacent to and in contact with the lateral and upstream sides of section 231. Section 231 can function to scatter and redirect neutrons that enter section 231 from central region 210 and/or target 60. The scattering function can include reducing the energy of fast neutrons at large forward angles such that the neutrons are at a suitable epithermal energy level if redirected back to central region 230 and/or beam output 202. Suitable materials for section 231 can be a combination of any of the S and R materials disclosed herein, but are not limited to such. Section 232 can function to redirect neutrons that enter section 232 such that those neutrons can be scattered by section 231 and central region 210. Section 232 can also function as a photon shield. Neutrons passing through both sections 231 and 232 are scattered by section 231 to allow for easier absorption in peripheral region 250. Suitable materials for section 232 can include any of the R materials disclosed herein, not limited to such. In other embodiments, sections 231 and 232 can be made relatively thinner than depicted in FIG. 6A and those sections can be repeated two or more times (e.g., 231-232-231-232, etc.) within intermediate region 230.

When used in combination with central region 210 having shell 208 as described with respect to FIGS. 4A-4K, redirection of neutrons by intermediate region sections 231 and 232 back into central region 210 results in those neutrons entering section 208. Section 208 can be configured with an S material or other material that acts as a low resistance pathway for propagation of those neutrons from intermediate region 230 to beam output 202. If central region 210 includes a section with an R material, such as section 211 in the example of FIGS. 4A-4C, then that material can assist in channeling the neutrons into shell 208, somewhat like a backstop for the neutrons redirected from region 230.

In FIG. 6B, intermediate region 230 again includes a layered arrangement of sections where each section is adjacent to and surrounds the lateral and upstream sides of the immediately preceding section. Section 231 is immediately adjacent central region 210, and can function as a neutron absorber, for example using any of the Ab materials disclosed herein. Section 232 is the next upstream section and can function as a photon absorber, for example using any of the R materials disclosed herein. Section 233 is the next upstream section and can function as epithermal scattering section, for example using any of the Epi-S materials disclosed herein. Section 234 is the next upstream section and can function as a neutron absorber, for example using any of the Ab materials disclosed herein. Section 235 is the next upstream section and can function as a photon absorber, for example using any of the R materials disclosed herein.

### Example Embodiments of Peripheral Regions

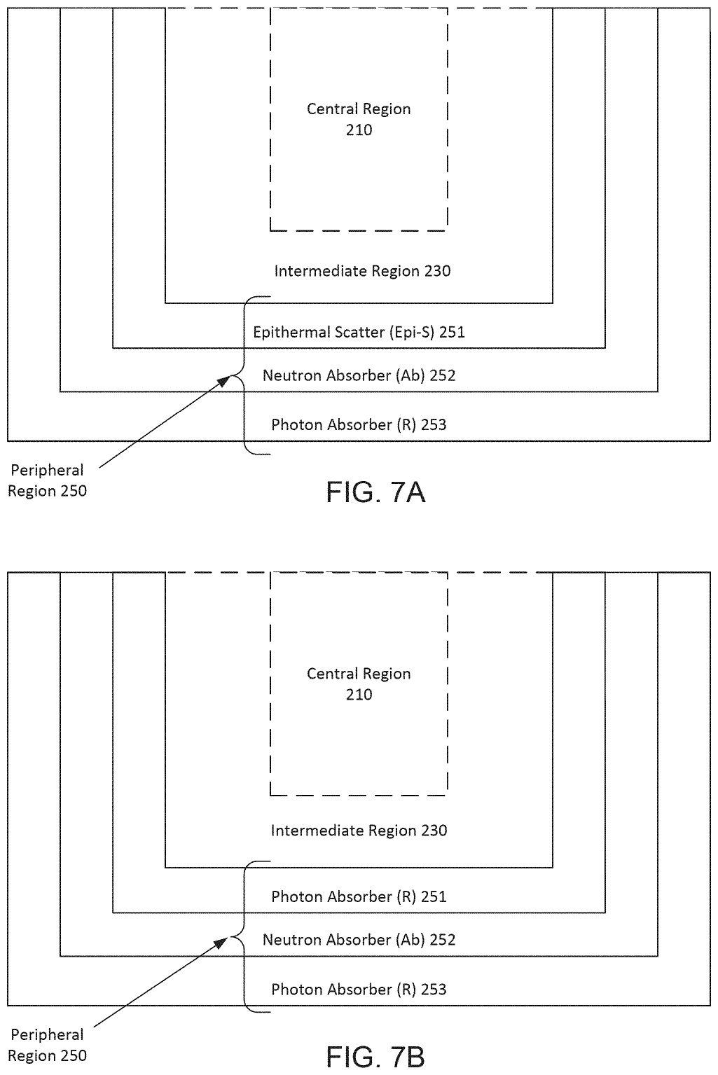

FIGS. 7A-7B are cross-sectional views depicting example embodiments of peripheral region 250. In both examples, region 250 includes a first section 251 adjacent to and in contact with the lateral and upstream sides of intermediate region 230. Region 250 also includes a second section 252 adjacent to and in contact with the lateral and upstream sides of section 251. Region 250 also includes a third section 253 adjacent to and in contact with the lateral and upstream sides of section 252.

In FIG. 7A, section 251 can function to scatter epithermal neutrons that enter section 251 from intermediate region 230. This reduces the energy of epithermal neutrons such that the neutrons are at a lower epithermal or thermal more readily absorbed by section 252, configured as a neutron absorber. Suitable materials for section 251 can be, for example, any of the Epi-S materials disclosed herein, and materials for section 252 can be, for example, any of the Ab materials disclosed herein. Section 253 can function as a photon shield to remove any remaining gamma radiation. Suitable materials for section 232 can include any of the R materials disclosed herein. FIG. 7B is similar to FIG. 7A except that section 251 is instead configured as a photon shield.

### Example Embodiments of Frontal Regions

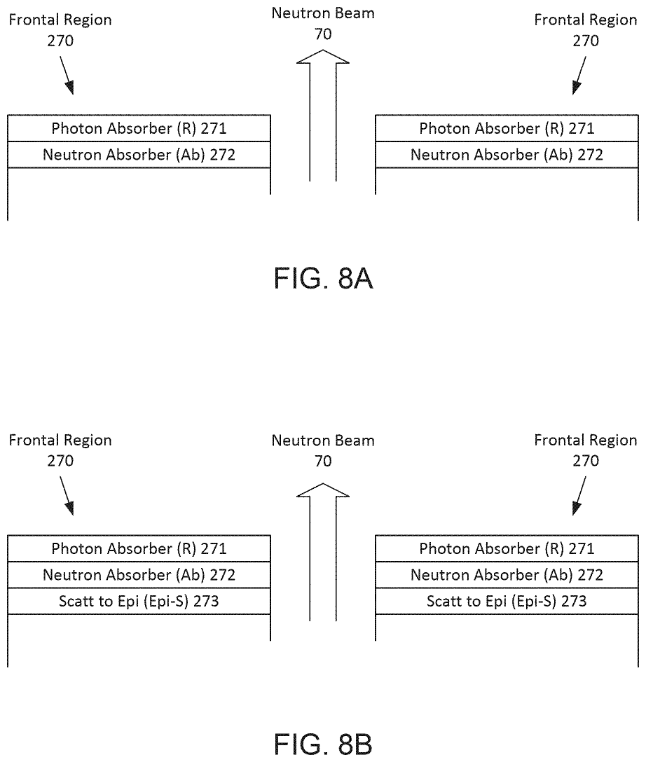

FIGS. 8A-8B are cross-sectional views depicting example embodiments of frontal region 270. Frontal region 270 can include more sections than those depicted here, as well as sections of different types or functions. Frontal region can extend across the entirety of the front side of NBC 200, except for recess 205. In both examples, region 270 includes a first section 271 that can form the front-most portion (e.g., the patient-facing portion) of NBC 200. A second section 272 is located upstream of section 271. Section 271 can function as a photon absorber and can be, for example, formed from any of the R materials disclosed herein. Section 272 can function as a neutron absorber and can be, for example, formed from any of the Ab materials disclosed herein. Together sections 271 and 272 filter or reduce any remaining photon and neutron radiation emanating from the frontal side of NBC 200 at locations around the desired beam output. In the embodiment of FIG. 8B, a third section 273 is located upstream of section 272. Section 273 can function as an epithermal scattering section, for example composed of any of the Epi-S materials disclosed herein, that acts to down scatter epithermal neutrons to a lower epithermal or thermal energy range where they are more readily absorbed by section 272.

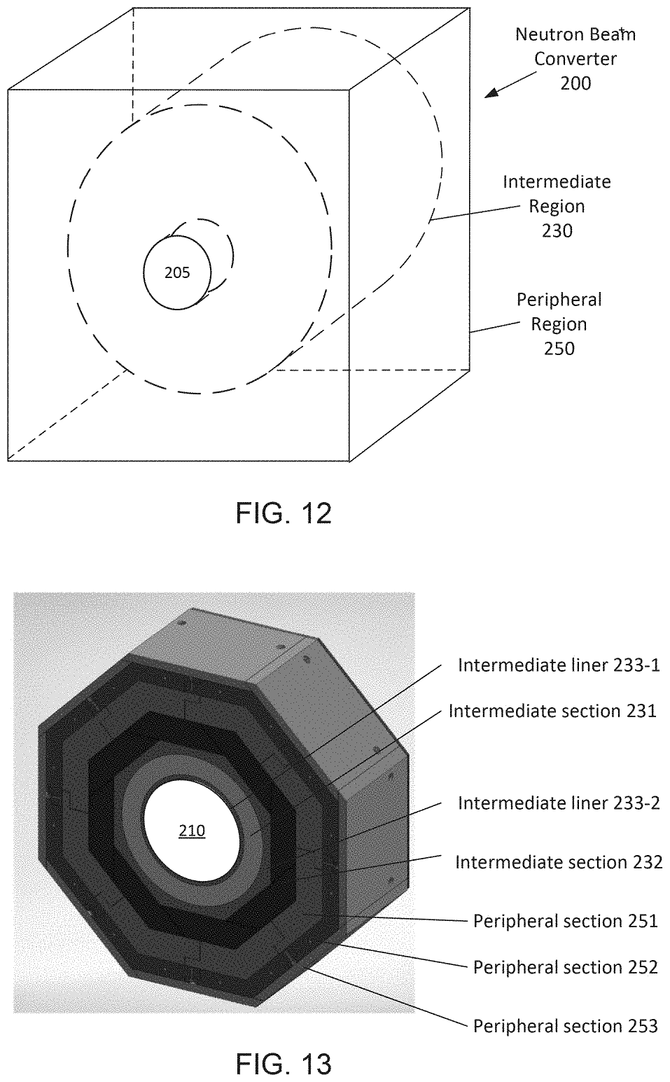

In some embodiments, placement of a titanium-boron alloy or boron carbide in frontal region 270, or in or near beam output 202 or recess 206), substantially eliminates the low energy tail in the emitted neutron spectrum of converted beam 70, which consequently increases the average cosine theta of beam 70 upon exit (e.g., makes the neutrons more forward-directed out of NBC 200), as scattering laws dictate that the most probable scattering angle goes from isotropic to more forward directed as neutron energy increases. Placement within peripheral region for Epi-S and Ab functions is also advantageous, Ti—B alloys such as titanium diboride and B—C alloys like boron carbide are very hard ceramics that are highly shapeable, and thus can be made into specialized shapes for NBC 200, such as the transition section described with respect to the embodiment of FIG. 12.

### Additional Example Embodiments of NBC 200

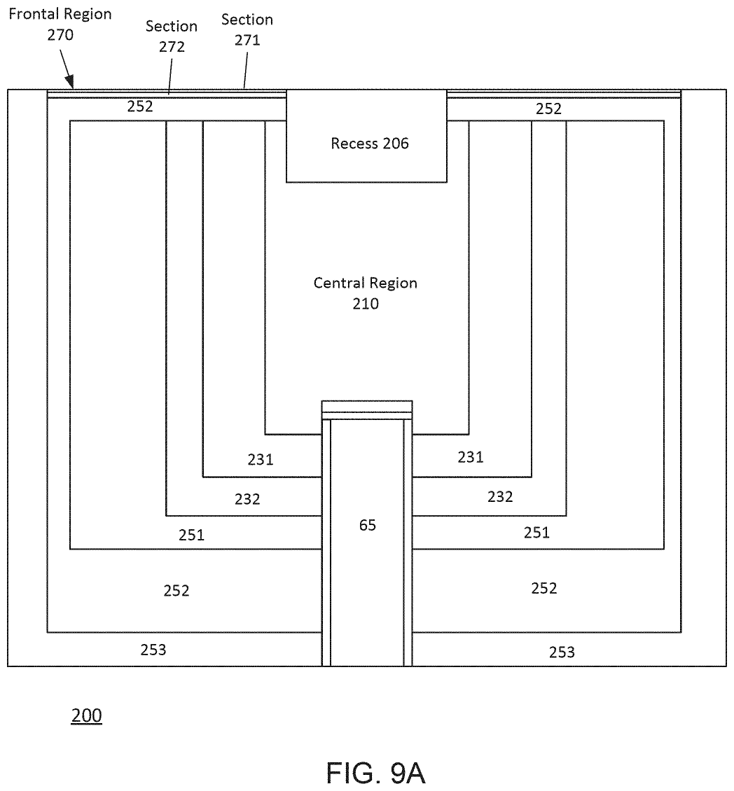

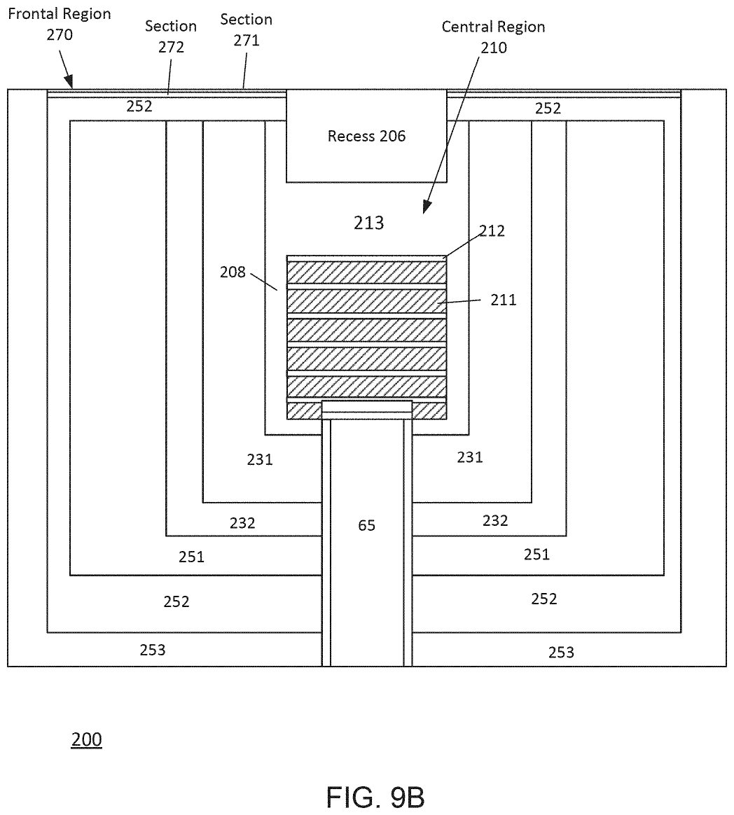

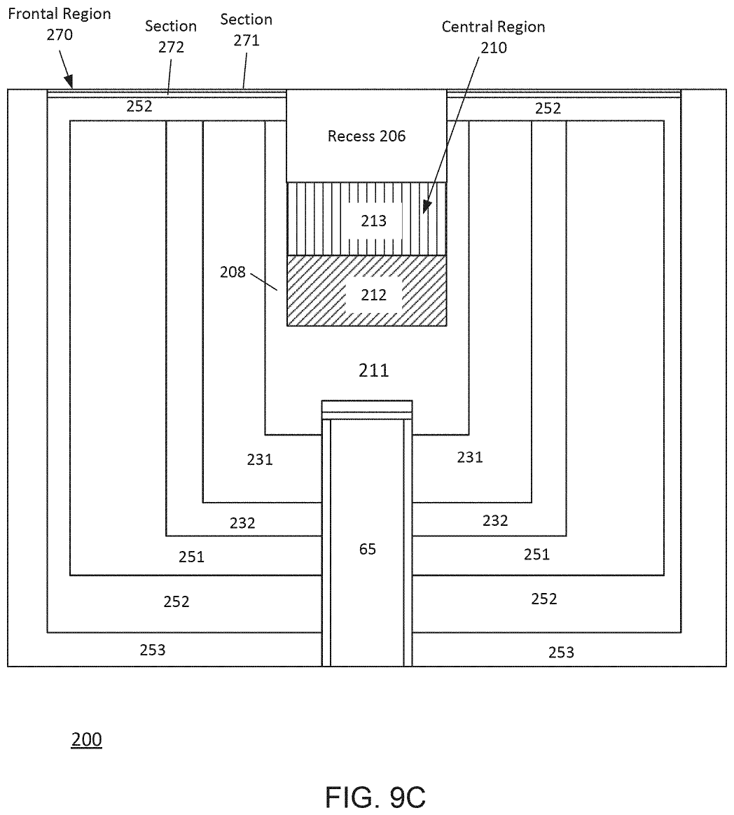

FIGS. 9A-9C are cross-sectional views depicting example embodiments of NBC 200 in a first configuration. These embodiments have similar construction with the only difference being the representation of central region 210. Each of the embodiments has an intermediate region having two sections 231 and 232. The sections can be configured in accordance with the embodiments of intermediate region 230 described herein. Each of the embodiments also has a peripheral region having three sections 251, 252, and 253, which can be configured in accordance with the embodiments of peripheral region 250 described herein. Frontal region 270 is depicted here as a combination of two sections 271, 272, which can be configured in accordance with the embodiments of frontal region 270 described herein. Central region 210 is depicted here in general format, and any of the embodiments of central region 210 described herein, including each of those of FIGS. 4A-4K, can be implemented here. Section 252 of the peripheral region extends across the downstream side of sections 251, 232, 231, and a portion of central region 210, in a position between these components and frontal region 270. When configured as a neutron absorber, section 252 can act to provide further absorption benefits across the front side of NBC 200 and locations where neutron emission is not desired.

In the embodiment of FIG. 9B, central region 210 includes shell 208 encompassing a layered region in an arrangement similar to that described with respect to FIG. 4I. The layered region includes a series of six layers composed of a combination of an R material and an S1 material (R+S1) depicted in crosshatch, with six layers of an S2 material interjected therein and thereon.

In the embodiment of FIG. 9C, central region 210 includes shell 208 holding several scattering sections similar to that described with respect to FIG. 4E. Shell 208 is composed of an S1 material that bridges across target assembly 65. A first section 211 it is composed of an S2 material, and a second section 212 located downstream of section 211 is composed of an S3 material.

Table 7 sets forth ten examples of material combinations that can be used for the embodiments of FIGS. 9B and 9C. In these ten examples central region has three sections 211, 212, and 213 with shell 208 having a unified composition with section 213. These examples are not exhaustive of all possible configurations for the embodiments of FIGS. 9B and 9C, as other materials and configurations are described herein.

TABLE 7
(Embodiments of FIGS. 9B, 9C)

| Region | Section | Example 1 | Example 2 | Example 3 | Example 4 | Example 5 |
|---|---|---|---|---|---|---|
| Central | 208/213 | Aluminum, | Aluminum, | Aluminum, | Aluminum, | Aluminum, |
| 210 | | Magnesium | Magnesium | Magnesium | Magnesium | Magnesium |
| | 211 | Bismuth, | Bismuth, | Magnesium, | Magnesium, | Lead, |
| | | Fluorine | Fluorine | Fluorine | Fluorine | Fluorine |
| | 212 | Aluminum | Aluminum | Aluminum | Aluminum | Beryllium |
| | | Oxide, | Oxide, | Oxide, | Oxide, | or |
| | | Carbide, or | Carbide, or | Carbide, or | Carbide, or | Beryllium |
| | | Nitride | Nitride | Nitride | Nitride | Oxide |
| Inter. | 231 | Bismuth, | Bismuth, | Bismuth, | Lead, | Lead, |
| 230 | | Fluorine | Fluorine | Fluorine | Fluorine | Fluorine |
| | 232 | Bismuth | Bismuth | Bismuth | Lead | Lead |
| Periph. | 251 | Titanium, | Titanium, | Titanium, | Titanium, | Titanium, |
| 250 | | Vanadium | Vanadium | Vanadium | Vanadium | Vanadium, |
| | | | | | | Aluminum |
| | 252 | Titanium, | Aluminum, | Titanium, | Titanium, | Titanium, |
| | | Boron | Magnesium | Boron | Boron | Boron |
| | 253 | Bismuth | Tungsten | Tungsten | Lead | Lead |
| Frontal | 271 | Tungsten | Tungsten | Tungsten | Lead | Lead |
| 270 | 272 | Lithium, | Lithium, | Titanium, | Titanium, | Titanium, |
| | | Titanium | Titanium | Boron | Boron | Boron |
| Region | Section | Example 6 | Example 7 | Example 8 | Example 9 | Example 10 |
| Central | 208/213 | Aluminum, | Aluminum, | Aluminum, | Aluminum | Aluminum |
| 210 | | Magnesium | Magnesium | Magnesium | | |
| | 211 | Bismuth, | Bismuth, | Lead, | Magnesium, | Magnesium, |
| | | Fluorine | Fluorine | Fluorine | Fluorine | Fluorine |
| | 212 | Beryllium | Beryllium | Beryllium | Metal | Metal |
| | | or | or | or | Carbide, | Carbide, |
| | | Beryllium | Beryllium | Beryllium | Nitride, or | Nitride, or |
| | | Oxide | Oxide | Oxide | Oxide | Oxide |
| Inter. | 231 | Lead, | Lead, | Lead, | Lead, | Bismuth, |
| 230 | | Fluorine | Fluorine | Fluorine | Fluorine | Fluorine |
| | 232 | Lead | Lead | Lead | Lead | Lead |
| Periph. | 251 | Titanium, | Titanium, | Titanium, | Titanium, | Titanium, |
| 250 | | Vanadium, | Vanadium, | Vanadium, | Vanadium, | Vanadium |
| | | Aluminum | Aluminum | Aluminum | Aluminum | |
| | 252 | Aluminum, | Aluminum, | Aluminum, | Boronated | Aluminum, |
| | | Magnesium | Magnesium | Magnesium | Polyethylene | Magnesium |
| | 253 | Boron, | Boron, | Boron, | Boron, | Lead |
| | | Carbon | Carbon | Carbon | Carbon | |
| Frontal | 271 | Lead | Lead | Lead | Lead | Lead |
| 270 | 272 | Boron, | Boron, | Boron, | Boron, | Titanium, |
| | | Carbon | Carbon | Carbon | Carbon | Boron |

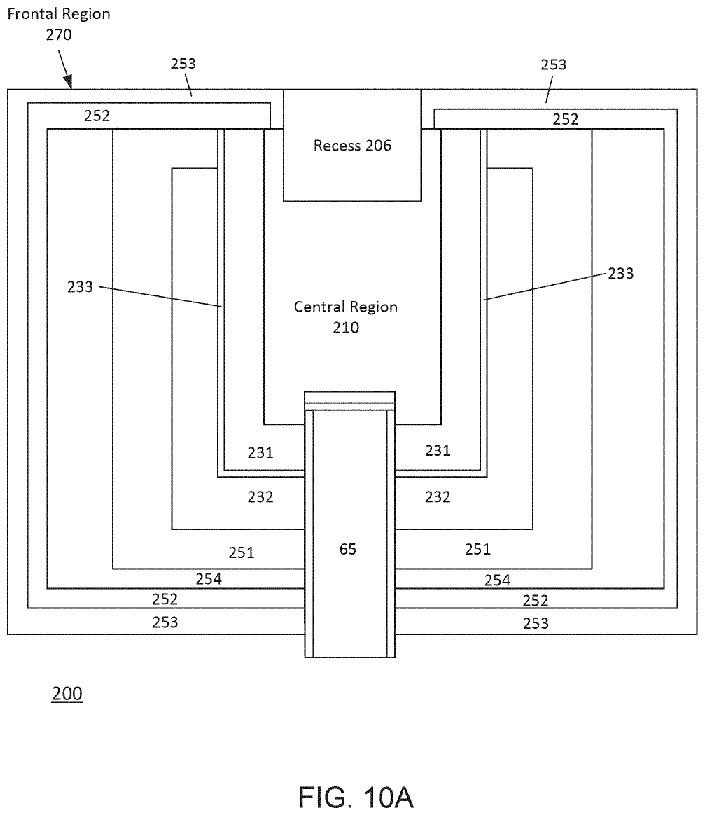

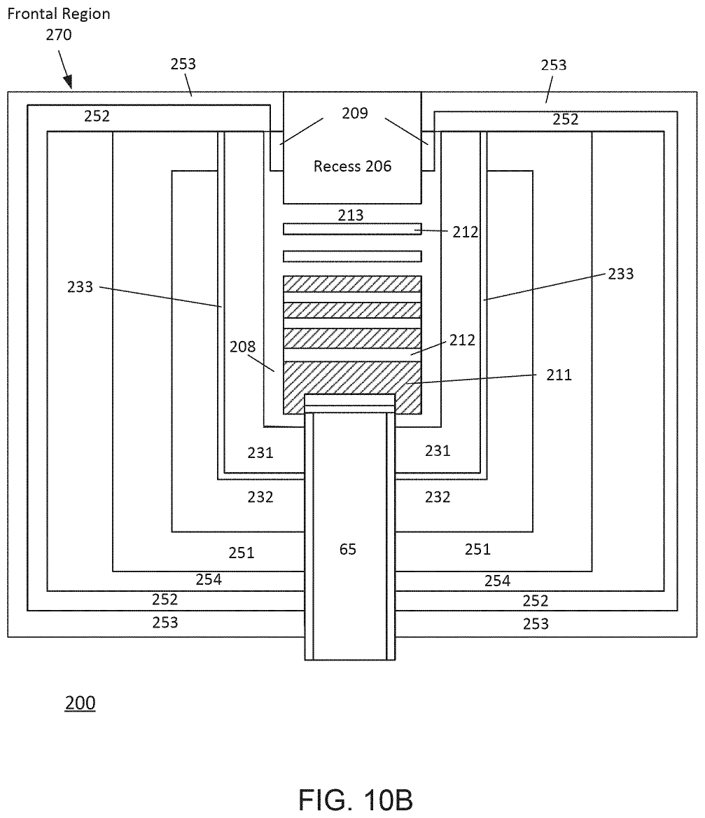

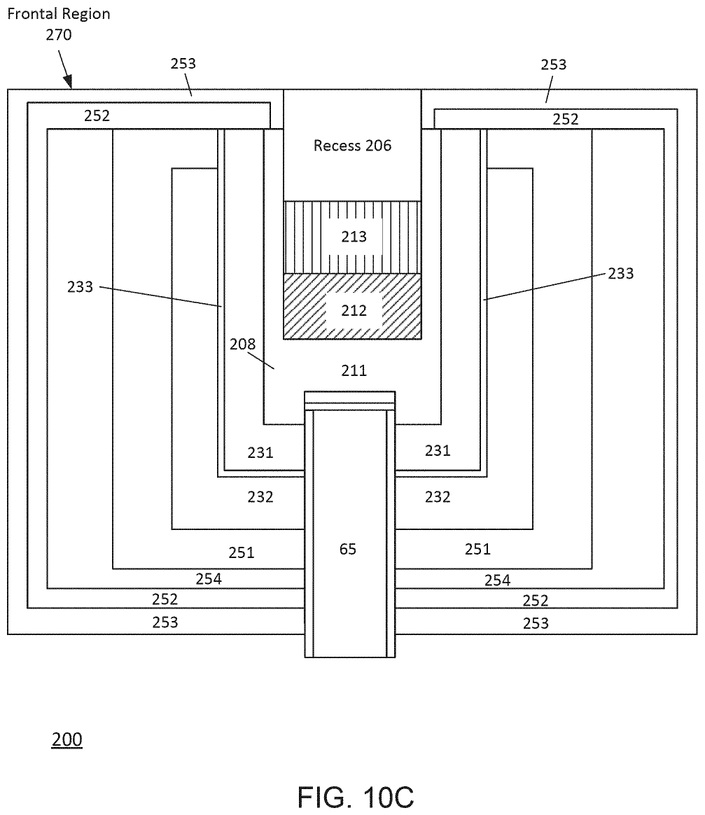

FIGS. 10A-10C are cross-sectional views depicting example embodiments of NBC 200 in a second configuration. These embodiments have similar construction with the only difference being the representation of central region 210. Each of the embodiments has an intermediate region having two sections 231 and 232 with a third section 233 interposed therebetween. Sections 231 and 232 can be configured in accordance with the embodiments of intermediate region 230 described herein. Section 233 can be configured as a containment vessel that functions with other components to maintain the position of sections 231 and/or 232 in place. Such a vessel can be desirable if the materials forming sections 231 and/or 232 are in powder form. A similar liner (not shown) can be placed between sections 231 and 208 if desired. Each of the embodiments also has a peripheral region having three sections 251, 252, and 253, which can be configured in accordance with the embodiments of peripheral region 250 described herein. An optional fourth section 254 can be interposed between sections 251 and 252 and can function, for example, for additional neutron absorption. Sections 252 and 253 can wrap around the frontal side of NBC 200 and function as frontal region 270. Section 253 continues and forms a flange or lip along an inner surface of recess 205 and can act as a neutron redirector in that location. Central region 210 is depicted here in general format, and any of the embodiments of central region 210 described herein, including each of those of FIGS. 4A-4K, can be implemented here.

In the embodiment of FIG. 10B, central region 210 includes shell 208 encompassing a layered region in an arrangement similar to that described with respect to FIG. 4G. The layered region includes a series of four sections 211 depicted in crosshatch, with three layers of section 212 interjected therein. Shell 208 can be formed of the same material as the third section 213 to make those portions contiguous. Two layers of section 212 are contained within section 213 downstream of the other layered section. Also in this embodiment a liner 209 is present around recess 205. Liner 209 can be composed of a redirector material or a scattering material.

In the embodiment of FIG. 10C, central region 210 includes shell 208 holding several scattering sections similar to that described with respect to FIG. 4E. Shell 208 is composed of the same material as section 211. Section 212 is located downstream of section 211 and section 213 is located downstream of section 212.

FIGS. 11A-11C are cross-sectional views depicting example embodiments of NBC 200 in a third configuration. These embodiments have similar construction with the only difference being the representation of central region 210. Each of the embodiments has an intermediate region having two sections 231 and 232, which can be configured in accordance with the embodiments of intermediate region 230 described herein. Each of the embodiments also has a peripheral region having three sections 251, 252, and 253, which can be configured in accordance with the embodiments of peripheral region 250 described herein. In this embodiment, section 251 has a nonuniform lateral dimension with an outermost surface that flares outward laterally at an axial position aligned with target 60. The inner surface of section 251 has a constant lateral dimension. A fourth section 254 is located around the outer lateral surface of section 251 and can function, for example, for additional neutron absorption. Sections 252 and 253 can wrap around the frontal side of NBC 200 and function as part of frontal region 270. Section 251 can also continue through frontal region at an oblique angle with respect to axis 203 and in a discontinuous configuration separated from the remainder of section 251 by platelike portions of sections 252 and 253. Section 254 can also continue through frontal region at an oblique angle with respect to axis 203 over the oblique sections 251 and also in a discontinuous configuration separated from the remainder of section 254 by platelike portions of sections 252 and 253. Central region 210 is depicted here in general format, and any of the embodiments of central region 210 described herein, including each of those of FIGS. 4A-4K, can be implemented here.

In the embodiment of FIG. 11B, central region 210 includes shell 208 encompassing a layered region in an arrangement similar to that described with respect to FIG. 4G. The layered region includes a series of four sections 211 depicted in crosshatch, with three layers of section 212 interjected therein. Shell 208 can be formed of the same material as the third section 213 to make those portions contiguous. Two layers of section 212 are contained within section 213 downstream of the other layered section. Also in this embodiment a liner 209 is present around recess 205. Liner 209 can be composed of a redirector material or a scattering material.

In the embodiment of FIG. 11C, central region 210 includes shell 208 holding several scattering sections similar to that described with respect to FIG. 4E. Shell 208 is composed of an S1 material that bridges across target assembly 65. Shell 208 is composed of the same material as section 211. Section 212 is located downstream of section 211 and section 213 is located downstream of section 212.

Table 8 sets forth ten examples of material combinations that can be used for the embodiments of FIGS. 10B, 10C, 11B, and 11C. In these ten examples central region has three sections 211, 212, and 213 with shell 208 having a unified composition with section 213. These examples are not exhaustive of all possible configurations for the embodiments of FIGS. 10B, 10C, 11, and 11C, as other materials and configurations are described herein.

TABLE 8
(Embodiments of FIGS. 10B, 10C, 11B, 11C)

| Region | Section | Example 1 | Example 2 | Example 3 | Example 4 | Example 5 |
|---|---|---|---|---|---|---|
| Central | 209 | Lead | Lead | Lead | Lead | Beryllium |
| 210 | | | | | | or |
| | | | | | | Beryllium |
| | | | | | | Oxide |
| | 211 | Bismuth, | Bismuth, | Magnesium, | Magnesium, | Lead, |
| | | Fluorine | Fluorine | Fluorine | Fluorine | Fluorine |
| | 212 | Aluminum | Aluminum | Aluminum | Aluminum | Beryllium |
| | | Oxide, | Oxide, | Oxide, | Oxide, | or |
| | | Carbide, or | Carbide, or | Carbide, or | Carbide, or | Beryllium |
| | | Nitride | Nitride | Nitride | Nitride | Oxide |
| | 208/213 | Aluminum, | Aluminum, | Aluminum, | Aluminum, | Aluminum, |
| | | Magnesium | Magnesium | Magnesium | Magnesium | Magnesium |
| Inter. | 231 | Bismuth, | Bismuth, | Bismuth, | Lead, | Lead, |
| 230 | | Fluorine | Fluorine | Fluorine | Fluorine | Fluorine |
| | 233 | Aluminum | Aluminum | Aluminum | Aluminum | Aluminum |
| | 232 | Bismuth | Bismuth | Bismuth | Lead | Lead |
| Periph. | 251 | Titanium, | Titanium, | Titanium, | Titanium, | Titanium, |
| 250 | | Vanadium | Vanadium | Vanadium | Vanadium | Vanadium, |
| | | | | | | Aluminum |
| | 254 | Lithium, | Lithium, | Boronated | Boronated | Boron, |
| | | Titanium | Titanium | Polyethylene | Polyethylene | Carbon |
| | 252 | Titanium, | Boron, | Titanium, | Boron, | Titanium, |
| | | Boron | Carbon | Boron | Carbon | Boron |
| | 253 | Bismuth | Tungsten | Tungsten | Lead | Lead |
| Region | Section | Example 6 | Example 7 | Example 8 | Example 9 | Example 10 |
| Central | 209 | Beryllium or | Beryllium | Beryllium | Lead | Lead |
| 210 | | Beryllium | or | or | | |
| | | Oxide | Beryllium | Beryllium | | |
| | | | Oxide | Oxide | | |
| | 211 | Bismuth, | Bismuth, | Lead, | Lead, | Bismuth, |
| | | Fluorine | Fluorine | Fluorine | Fluorine | Fluorine |
| | 212 | Beryllium or | Beryllium | Beryllium | Metal | Metal |
| | | Beryllium | or | or | Carbide, | Carbide, |
| | | Oxide | Beryllium | Beryllium | Nitride, or | Nitride, or |
| | | | Oxide | Oxide | Oxide | Oxide |
| | 208/213 | Magnesium, | Aluminum, | Aluminum, | Aluminum, | Aluminum |
| | | Fluorine | Magnesium | Magnesium | Magnesium | |
| Inter. | 231 | Lead, | Lead, | Lead, | Lead, | Bismuth, |
| 230 | | Fluorine | Fluorine | Fluorine | Fluorine | Fluorine |
| | 233 | Aluminum | Aluminum, | Aluminum | Aluminum, | Aluminum |
| | | | Magnesium | | Magnesium | |
| | 232 | Lead | Lead | Lead | Lead | Lead |
| Periph. | 251 | Titanium, | Titanium, | Titanium, | Titanium, | Titanium, |
| 250 | | Vanadium, | Vanadium, | Vanadium, | Vanadium, | Vanadium |
| | | Aluminum | Aluminum | Aluminum | Aluminum | |
| | 254 | Lithium, | Boron, | Boron, | Boron, | Titanium, |
| | | Titanium | Carbon | Carbon | Carbon | Boron |
| | 252 | Aluminum, | Aluminum, | Aluminum, | Boronated | Aluminum, |
| | | Magnesium | Magnesium | Magnesium | Polyethylene | Magnesium |
| | 253 | Boron, | Boron, | Boron, | Boron, | Lead |
| | | Carbon | Carbon | Carbon | Carbon | |

### Example Embodiments Having Cylindrical and/or Non-Cylindrical Surfaces

Certain materials exhibit excellent properties for use in neutron beam conditioning and shielding, while at the same time being shapeable to facilitate construction of NBC 200 and the surrounding support structure. For example, the aforementioned embodiments of NBC 200 having various regions and sections that are cylindrical, or substantially cylindrical (e.g., being cylinder-like around a beam), having a curved outer surface that might be prone to roll, shift, or is otherwise not as convenient to work with and install as a flat surface. FIG. 12 is a perspective view depicting an example embodiment of NBC 200 similar to the embodiment of FIG. 3A, where the outermost surface of the peripheral region 250 is multi-sided flat-faced shape, such as a right rectangular prism or cube. A section of peripheral region 250 can be configured with an lateral inner surface having a curved or an at least partly cylindrical shape corresponding to an lateral outer curved or at least party cylindrical surface of intermediate region 230. That interior section can be configured with a lateral outer surface having a multi-sided flat-faced shape and thus can be used to transition the sections of NBC 200 from generally cylindrical ones to those with flat faces.

Any of the sections of peripheral region 250 can be used to perform this transition, including the innermost section (e.g., section 251), the outermost section (e.g., section 253), and sections located therebetween (e.g., sections 252 and 254). The transition is facilitated if the transition section is composed of a material that can be easily shaped, molded, machined, and/or 3D printed. Examples of such materials are ceramic titanium diboride, ceramic boron carbide, polymers like polyethylene (PE) (e.g., boron-doped polyethylene of section 254) and ETFE. The materials of sections placed outside of the transition section can then be configured as blocks or plates without significant curved surfaces that are relatively easy to manufacture and assemble into NBC 200's complete structure.

FIG. 13 is a perspective view depicting another example embodiment of NBC 200 similar to the embodiment of FIGS. 9A-10C, having central region 210, inner intermediate liner 233-1, intermediate section 231, outer intermediate liner 233-2, intermediate section 232, and peripheral sections 251, 252, and 253. Here intermediate liner 233-3 is the transition section between a generally curved lateral outer surface or shape and a multi-sided lateral inner surface or shape. The innermost surface (interior) of intermediate liner 233-2 can have a curvature or rounding that corresponds to (e.g., matches) the curvature or rounding of the outer surface of intermediate section 231. The outermost surface (exterior) of intermediate liner 233-2 can be a polygonal surface that matches the innermost surface of intermediate section 232. In the depicted embodiment, region 210 and the sections 231-1 and 231 interior to liner 233-2 have generally cylindrical shapes, while the sections 232 and sections 251, 252, and 253 of peripheral region 250 have generally polygonal surfaces. Similar to the embodiment of FIG. 12, this configuration permits optimal shaping (e.g., cylindrical) for neutron conversion in the innermost volume of NBC 200, while transitioning to the polygonal surface configurations in the outermost volume of NBC 200 where neutron conversion requires less symmetry and concerns relating to ease of assembly, manufacture, and maintenance preside. While shown here as a polygon with eight lateral surfaces or sides, in other embodiments the polygon can have four, five, six, seven, nine, ten, or more lateral surfaces, plus at least two end surfaces (at least one corresponding to the upstream-most location and at least one corresponding to the downstream-most location).

The cylindrical and polygonal lateral sides or shapes need not be perfect geometrical cylinders or polygons. Those of ordinary skill in the art, upon reading this description, will recognize that such sides or shapes can be substantially cylindrical or polygonal, and those of ordinary skill in the art will readily recognize sides or shapes that are substantially cylindrical or polygonal. For example, some flatness can be present on the lateral cylinder sides or shapes, where that flatness has a negligible effect on the overall neutron beam profile output from the beam output. Similarly, some roundness or curvature can be present on the lateral polygonal sides or shapes, where that roundness or curvature also has a negligible effect on the overall neutron beam profile output from the beam output. In some embodiments, a substantially cylindrical shape varies by no more than 5% of the overall lateral dimension from a geometric cylinder. In some embodiments, a substantially polygonal shape varies by no more than 5% of the overall lateral dimension from the corresponding geometric polygon.

NBC 200, including each section of regions 210, 230, 250, and 270 can be fabricated according to the process that provides the desired balance between manufacturability, performance, and cost. Portions of NBC 200 making up a section one of regions 210, 230, 250, and 270 can be fabricated separately and then assembled to form the larger section, or even larger region. The materials for NBC 200 can be in contiguous solid form (e.g., a sheet of lead). The combination of first and second materials, such as R and S materials or different types of S materials (e.g., Fast-S and NR-S materials), can be in the form of a bonded chemical compound. Elementary materials and combined materials can be in a raw granular or powdered form and then set into the desired shape through casting, molding, sintering, or mixing with additives (e.g., adhesives like epoxy or enamel blends, etc.). The materials can also be in granular or powdered form and maintained or held in the desired shape using a metallic housing or casement.

Various aspects of the present subject matter are set forth below, in review of, and/or in supplementation to, the embodiments described thus far, with the emphasis here being on the interrelation and interchangeability of the following embodiments and the aspects thereof. In other words, an emphasis is on the fact that each aspect of the embodiments can be combined with each and every other aspect unless explicitly stated or taught otherwise.

In a first group of embodiments, a neutron beam converter is provided, the converter including: a beam input configured to receive neutrons from a neutron generation target; a beam output configured to output neutrons; a central region traversed by an axis between the beam input and the beam output, where the central region is configured to scatter neutrons to a treatment energy range; an intermediate region located laterally around the central region and configured to redirect and scatter neutrons, where the intermediate region includes a first intermediate section and a second intermediate section each including a different material; and a peripheral region located laterally around the intermediate region and configured to absorb neutrons and gamma radiation, where the peripheral region includes a first peripheral section and a second peripheral section each including a different material.

In some embodiments of the first group, the central region can include a first section, a second section, and a third section, where each of the first, second, and third sections include a different material, and where each of the first, second, and third sections are traversed by the axis. The first section can include fluorine, the second section can include magnesium, and the third section can include aluminum.

In some embodiments of the first group, the central region can include a first material and a second material. The first material can include aluminum and the second material can include magnesium. The first material can be configured to scatter generated neutrons towards or into a treatment energy range and configured to redirect generated neutrons, and the second material can be configured to non-resonantly scatter generated neutrons. The first material can include lead and fluorine. The second material can include beryllium. The second material can include at least one of an oxide, a carbide, or a nitride. The second material can be configured as a layer between a first central section including the first material and a second central section including the first material. The first central section, the layer, and the second central section can be traversed by the axis. The central region can include a third material that includes at least one of: aluminum, fluorine, and magnesium.

In some embodiments of the first group, the central region can include a cylindrical portion that extends from the beam input to the beam output. The cylindrical portion can have a first terminus upstream of an installed location of the neutron generation target. The cylindrical portion can have a first terminus along an upstream-most face of the neutron beam converter. The cylindrical portion can have a second terminus in proximity with the beam output. The cylindrical portion can be configured to act as a low resistance pathway for neutrons relative to the intermediate region. The cylindrical portion can include a tubular portion. The cylindrical portion can be configured to act as a low resistance pathway for neutrons relative to the intermediate region and a material within the tubular portion. The tubular portion can include at least one of: aluminum, fluorine, and magnesium, and laterally surrounds a first central section including a first material and a second material. The cylindrical portion can include at least one of: aluminum, fluorine, and magnesium. The cylindrical portion can traverse the axis.

In some embodiments of the first group, the converter can have a downstream-most surface and a recess from the downstream-most surface at the beam output. The recess can have a sidewall portion including a neutron redirector. The neutron redirector can include at least one of lead, nickel, bismuth, and tungsten. The recess can be cylindrical and the converter can include a tubular liner including beryllium, the tubular liner located in proximity with a sidewall of the recess.

In some embodiments of the first group, the central region can include: a first central section traversed by the axis and configured to scatter neutrons to the treatment energy range; a second central section traversed by the axis, located downstream of the first central section, and configured to scatter neutrons in an epithermal energy range to lower energies; and a third central section traversed by the axis, located downstream of the second central section, and configured to absorb neutrons scattered to the lower energies.

In some embodiments of the first group, the central region can include a solid section spaced apart from an installation location of the neutron generation target, where the solid section can be traversed by the access and located downstream of the installation location of the target. The solid section can be spaced apart from the installation location of the target by at least 10 centimeters.

In some embodiments of the first group, the first intermediate section can be relatively closer to the central region than the second intermediate section, and the first intermediate section can be configured to scatter generated neutrons and the second intermediate section is configured to redirect generated neutrons. The second intermediate section can be configured to redirect generated neutrons and absorb photons. The first intermediate section can be further configured to redirect neutrons. The first intermediate section can include at least one of fluorine, aluminum, and magnesium, and the second intermediate section can include at least one of lead, nickel, bismuth, and tungsten. The first intermediate section can include lead and the second intermediate section can include lead.

In some embodiments of the first group, the first intermediate section can be located laterally around the central region and the second intermediate section can be located laterally around the first intermediate section. The first intermediate section can be in contact with the central region, and the second intermediate section can be in contact with the first intermediate section.

In some embodiments of the first group, the intermediate region can extend laterally across a rear face of the central region.

In some embodiments of the first group, the second intermediate section can be located laterally around the first intermediate section, and the first intermediate section can be configured to scatter neutrons and the second intermediate section can be configured to redirect neutrons and absorb gamma radiation. The intermediate region can include a third intermediate section located laterally around the second intermediate section, where the third intermediate section can be configured to scatter neutrons from a first energy in an epithermal energy range to a second energy lower than the first energy. The intermediate region can include a fourth intermediate section located laterally around the third intermediate section, where the fourth intermediate section can be configured to absorb neutrons. The intermediate region can include a fifth intermediate section located laterally around the fourth intermediate section, where the fifth intermediate section can be configured to absorb gamma radiation.

In some embodiments of the first group, the first peripheral section can be relatively closer to the intermediate region than the second peripheral section, and the first peripheral section can be configured to absorb neutrons and the second peripheral section can be configured to absorb photons. The peripheral region can include a third peripheral section located relatively closer to the intermediate section than the first peripheral section, where the third peripheral section can be configured to scatter epithermal neutrons. The peripheral region can include a third peripheral section located relatively closer to the intermediate section than the first peripheral section, where the third peripheral section can be configured to absorb photons. The first and second peripheral sections can include titanium. The first peripheral section can include titanium and vanadium. The second peripheral section can include boron. The first peripheral section can be located laterally around the intermediate and central regions and the second peripheral section can be located laterally around the first peripheral section. The first peripheral section can be in contact with the intermediate region, and the second peripheral section can be in contact with the first peripheral section. The peripheral region can extend laterally across a portion of the rear of the central region and a portion of the rear of the intermediate region.

In some embodiments of the first group, the converter includes a frontal region extending laterally across the converter. The frontal region can be configured to absorb neutrons and photons. The frontal region can include a first frontal section configured to absorb photons and a second frontal section configured to absorb neutrons. The first frontal section can be located downstream of the second frontal section. The first frontal section can include an aperture for a recess at the beam output. The first frontal section can include at least one of lead, nickel, bismuth, and tungsten. The second frontal section can include at least one of lithium, cadmium, boron, titanium, gadolinium, indium, hafnium, and a hydrogenous polymer. The converter can include a third frontal section configured to scatter epithermal neutrons. The third frontal section can be located upstream of the second frontal section. The frontal region can form a continuation of the peripheral region.

In a second group of embodiments, a neutron beam converter is provided, the converter including: a beam input configured to receive neutrons; a beam output configured to output neutrons; a first region traversed by an axis between the beam input and the beam output; and a second region located laterally around the first region, where the second region includes an alloy including titanium and vanadium.

In some embodiments of the second group, the second region can include: a first section including the alloy; and a second section including boron. The first section can be located laterally around the first region and the second section can be located laterally around the first section. The converter can include a third region interposed between the first region and the second region, where the first region is a central region, the third region is an intermediate region, and the second region is a peripheral region. The second region can include lead. The second region can include a hydrogenous polymer.

In a third group of embodiments, a neutron beam converter is provided, the converter including: a beam input configured to receive neutrons; a beam output configured to output neutrons; a first region traversed by an axis between the beam input and the beam output; and a second region located either laterally around the first region or along a frontal region proximate to a treatment room, where the second region includes boron.

In some embodiments of the third group, the second region can include: a first section including an alloy of titanium and vanadium; and a second section including boron. The first section can be located laterally around the first region and the second section can be located laterally around the first section. The converter can include a third region interposed between the first region and the second region, where the first region is a central region, the third region is an intermediate region, and the second region is a peripheral region. The boron can be in ceramic titanium diboride or ceramic boron carbide.

In some embodiments of the third group, the second region includes a hydrogenous polymer.

In some embodiments of the third group, the converter includes the frontal region having boron, where the frontal region is located across a downstream front side of the converter.

In some embodiments of the third group, the second region exhibits an inverse velocity cross-section over a thermal and at least part of an epithermal neutron energy range.

In some embodiments of the third group, the second region exhibits a resonance peak cross-section in an epithermal neutron energy range.

In some embodiments of the third group, the second region is located laterally around the first region.

In some embodiments of the third group, the second region is located along the frontal region.

In a fourth group of embodiments, a neutron beam converter is provided, the converter including: a beam input configured to receive neutrons; a beam output configured to output neutrons; and a central region traversed by an axis between the beam input and the beam output, where the central region includes a plurality of first sections and a plurality of second sections, where the plurality of first sections is traversed by the axis and configured to resonantly down scatter neutrons, and where the plurality of second sections is traversed by the axis and configured to non-resonantly down scatter neutrons.

In some embodiments of the fourth group, the plurality of first sections and the plurality of second sections are in a stacked alternating arrangement.

In some embodiments of the fourth group, the plurality of second sections can include beryllium.

In some embodiments of the fourth group, the plurality of first sections can include at least one of magnesium, aluminum, and fluorine.

In some embodiments of the fourth group, the plurality of first sections can include lead and fluorine.

In some embodiments of the fourth group, the converter can include a plurality of third sections traversed by the axis and configured to resonantly down scatter neutrons, where the plurality of first sections includes a different material than the plurality of third sections. At least a portion of the plurality of second sections and the plurality of third sections can be in a stacked alternating arrangement.

In some embodiments of the fourth group, the converter includes a plurality of third sections traversed by the axis and configured to resonantly down scatter neutrons, where the plurality of first sections is interleaved with the plurality of second sections and the plurality of third sections, where the plurality of first sections includes at least one of magnesium, aluminum, or fluorine, where the plurality of second sections includes beryllium, and where the plurality of third sections includes a material including lead and fluorine.

In some embodiments of the fourth group, the plurality of first sections can be continuous.

In a fifth group of embodiments, a neutron beam converter is provided, the converter including: a beam input configured to receive neutrons; a beam output configured to output neutrons; a central region between the beam input and the beam output; an intermediate region located laterally around the central region; and a peripheral region located laterally around the intermediate and central regions, where the central region includes a first material including lead and fluorine, where the first material is traversed by an axis between the beam input and beam output, where the intermediate region includes a second material including lead and fluorine, and where a third material is located between the first material of the central region and the second material of the intermediate region.

In some embodiments of the fifth group, the first and second materials are the same.

In some embodiments of the fifth group, the first and second materials can be lead fluoride.

In some embodiments of the fifth group, the third material can include at least one of magnesium and aluminum.

In some embodiments of the fifth group, the third material can be a magnesium-aluminum alloy.

In some embodiments of the fifth group, the intermediate region includes: a first section having the second material; and a second section having a fourth material that includes at least one of lead, nickel, bismuth, and tungsten. The second section can be located laterally around the first section.

In some embodiments of the fifth group, the peripheral region can include at least one of lead, nickel, bismuth, and tungsten.

In a sixth group of embodiments, a neutron beam converter is provided, the converter including: a beam input configured to receive neutrons from a neutron generation target; a beam output configured to output neutrons; and a central region between the beam input and the beam output, where the central region includes at least one section including beryllium.

In some embodiments of the sixth group, the section including beryllium is separated from the beam input by another material.

In some embodiments of the sixth group, the at least one section is configured as a plate, where a central axis of the converter extending from the beam input to the beam output is normal to the plate.

In some embodiments of the sixth group, the at least one section is one of a plurality of first sections located at intervals between the beam input and the beam output, where each of the plurality of first sections includes beryllium.

In some embodiments of the sixth group, each adjacent pair of first sections in the plurality of first sections is separated by one of a plurality of second sections. Each of the plurality of second sections can be configured to resonantly scatter neutrons to a lower energy.

In some embodiments of the sixth group, the plurality of first sections can be configured as plates, where a central axis of the converter extending from the beam input to the beam output is normal to each first section of the plurality of first sections.

In some embodiments of the sixth group, the at least one section can include beryllium oxide.

In some embodiments of the sixth group, the at least one section can be configured to non-resonantly down scatter neutrons to a lower energy.

In a seventh group of embodiments, a neutron beam converter is provided, the converter including: a beam input configured to receive neutrons from a neutron generation target; a beam output configured to output neutrons; a first region between the beam input and the beam output; and a second region located laterally around the first region, where the second region includes a first section having an outer diameter that varies along a length of the first section.

In some embodiments of the seventh group, the first section has a maximum diameter at a first location aligned with a neutron generation target installation location. The diameter of the first section can decrease from the first location to a second location at a downstream terminus of the first section. The diameter of the first section can decrease from the first location to a second location at an upstream terminus of the first section.

In some embodiments of the seventh group, the first region is a central region, the second region is a peripheral region, and the converter includes an intermediate region between the central and the peripheral regions.

In some embodiments of the seventh group, an inner surface of the first section has a cylindrical shape with uniform diameter.

In some embodiments of the seventh group, the first section can be configured to down scatter epithermal neutrons.

In some embodiments of the seventh group, the first section can include titanium.

In some embodiments of the seventh group, the second region can have a cylindrical outer surface of uniform diameter.

In some embodiments of the seventh group, the second region includes a second section located laterally around the first section, where the second section has an inner diameter that varies corresponding to the outer diameter of the first section. The second section can have an outer diameter that is uniform along a length of the second section.

In an eighth group of embodiments, a neutron beam converter is provided, the converter including: a beam input configured to receive neutrons; a beam output configured to output neutrons; and a central region between the beam input and the beam output, where the central region includes a plurality of first sections in a stacked alternating arrangement with a plurality of second sections, where a first one of the plurality of first sections has a thickness relatively less than a thickness of a second one of the plurality of first sections.

In some embodiments of the eighth group, each of the plurality of first sections can have a different thickness.

In some embodiments of the eighth group, the thickness of the first one of the plurality of first sections is at least 10% less than the thickness of the second one of the plurality of first sections.

In some embodiments of the eighth group, the first one of the plurality of first sections can be located relatively closer to the beam output than the second one of the plurality of first sections.

In some embodiments of the eighth group, the first one of the plurality of first sections can be located relatively farther from the beam output than the second one of the plurality of first sections.

In some embodiments of the eighth group, thicknesses of the plurality of first sections can incrementally decrease in an upstream to downstream direction.

In some embodiments of the eighth group, thicknesses of the plurality of first sections can incrementally increase in an upstream to downstream direction.

In some embodiments of the eighth group, the plurality of first sections can have a different composition than the plurality of second sections.

In some embodiments of the eighth group, each of the plurality of first sections can be configured as plates.

In some embodiments of the eighth group, each of the plurality of first sections can be configured to non-resonantly scatter neutrons and each of the plurality of second sections can be configured to resonantly scatter neutrons.

In some embodiments of the eighth group, each of the plurality of first sections can be configured to resonantly scatter neutrons and each of the plurality of second sections can be configured to non-resonantly scatter neutrons.

In some embodiments of the eighth group, every one of the plurality of second sections can have the same thickness.

In some embodiments of the eighth group, each of the plurality of second sections can be thicker than each of the plurality of first sections.

In a ninth group of embodiments, a neutron beam converter is provided, the converter including: a beam input configured to receive neutrons; a beam output configured to output neutrons; and a central region between the beam input and the beam output, where the central region includes a plurality of first sections interleaved with a plurality of second sections, where the plurality of first sections includes a non-planar first section.

In some embodiments of the ninth group, the non-planar first section can have a cross-sectional profile that is chevron-shaped. The chevron-shaped cross-sectional profile can have an open side and a relatively more pointed side, where the relatively more pointed side faces the beam input. The chevron-shaped cross-sectional profile can have an open side and a relatively more pointed side, where the relatively more pointed side faces the beam output.

In some embodiments of the ninth group, each of the plurality of first sections can have a cross-sectional profile that is chevron-shaped.

In some embodiments of the ninth group, the non-planar first section can have a cross-sectional profile that is curved. The curved cross-sectional profile can have a concave side and a convex side, where the convex side faces the beam input. The curved cross-sectional profile can have a concave side and a convex side, where the concave side faces the beam input.

In some embodiments of the ninth group, each of the plurality of first sections can have a cross-sectional profile that is curved.

In some embodiments of the ninth group, the non-planar first section can be bowl-shaped.

In some embodiments of the ninth group, the non-planar first section can have a cross-sectional profile with a non-uniform thickness.

In some embodiments of the ninth group, the non-planar first section can have a cross-sectional profile that is curved, and the plurality of first sections can include a chevron-shaped first section.

In a tenth group of embodiments, a neutron beam converter is provided, the converter including: a beam input configured to receive neutrons from a neutron generation target; a beam output configured to output neutrons; a first region or section between the beam input and the beam output, where the first region or section has a first lateral outer surface that is curved around an axis between the beam input and beam output; and a second region or section located laterally around the first region or section, where the second region or section has a second lateral outer surface that is substantially multi-sided.

In some embodiments of the tenth group, the first lateral outer surface can be substantially cylindrical or cylindrical.

In some embodiments of the tenth group, the first region or section can be a first section of a central region of the converter. The second region or section can be a second section of the central region of the converter.

In some embodiments of the tenth group, the first region or section can be a central region of the converter. The second region or section is a section of an intermediate region of the converter. The second region or section is an intermediate region of the converter. The intermediate region can be configured to redirect and scatter generated neutrons.

In some embodiments of the tenth group, the second region or section can be a section of a peripheral region of the converter, and where the converter includes an intermediate region located between the central region and the peripheral region.

In some embodiments of the tenth group, the second region or section can be a peripheral region of the converter, and where the converter includes an intermediate region located between the central region and the peripheral region.

In some embodiments of the tenth group, the central region can be configured to scatter generated neutrons towards or into a treatment energy range.

In some embodiments of the tenth group, the second lateral outer surface can have four or more lateral sides and at least two end sides.

In some embodiments of the tenth group, the second lateral outer surface can have six or more lateral sides and at least two end sides.

In some embodiments of the tenth group, the second region or section can include a lateral inner side that corresponds in curvature to the first lateral outer side.

In some embodiments of the tenth group, the converter includes: two or more first sections of a central region, the two or more first sections of the central region each having a lateral outer side that is curved around the axis between the beam input and beam output; and two or more second sections of an intermediate region and/or a peripheral region of the converter, the two or more second sections each having a lateral outer side that is substantially multi-sided around the axis.

In the embodiments of the first, second, third, fourth, fifth, sixth, seventh, eighth, ninth, and tenth groups, the converter can be configured to output a neutron beam in an epithermal energy range from the beam output.

In the embodiments of the first, second, third, fourth, fifth, sixth, seventh, eighth, ninth, and tenth groups, the converter can be configured to output a neutron beam in an energy range from one electron volt to thirty kiloelectron volts. The converter can be configured to output the neutron beam with a peak neutron distribution and an average energy between 10 kiloelectron volts and 30 kiloelectron volts. The converter can be configured to output the neutron beam with at least 90% of the neutrons in the energy range from one electron volt to thirty kiloelectron volts.

In the embodiments of the first, second, third, fourth, fifth, sixth, seventh, eighth, ninth, and tenth groups, the converter can be configured to output a neutron beam in an energy range from one electron volt to ten kiloelectron volts. The converter can be configured to output the neutron beam with a peak neutron distribution and an average energy between three kiloelectron volts and ten kiloelectron volts. The converter can be configured to output the neutron beam with at least 90% of the neutrons in the energy range from one electron volt to ten kiloelectron volts.

In the embodiments of the first, second, third, fourth, fifth, sixth, seventh, eighth, ninth, and tenth groups, the converter can be configured for use in a boron neutron capture therapy system.

In the embodiments of the first, second, third, fourth, fifth, sixth, seventh, eighth, ninth, and tenth groups, the converter can include a neutron generation target. The neutron generation target can include lithium and can be configured to generate neutrons according to the reaction p+7Li→n+7Be. The neutron generation target can be configured to generate neutrons from a proton beam having an energy in the range of 1.9 to 3.0 megaelectron volts.

In the embodiments of the first, second, third, fourth, fifth, sixth, seventh, eighth, ninth, and tenth groups, the converter can be configured to receive a raw neutron beam from the neutron generation target and output a converted neutron beam, where the converted neutron beam has a relatively more focused forward direction than the raw neutron beam, a variation in intensity that is relatively less than the raw neutron beam, and a variation in energy that is relatively less than the raw neutron beam.

In an eleventh group of embodiments, a method of converting a neutron beam is provided, the method including: propagating a charged particle beam at a neutron generation target located within a neutron beam converter such that a neutron beam is emitted from a neutron beam output of the neutron beam converter, where the neutron beam converter is configured in accordance with any embodiment of the first, second, third, fourth, fifth, sixth, seventh, eighth, ninth, and tenth groups of embodiments.

In a twelfth group of embodiments, a method of treating a patient with boron neutron capture therapy (BNCT) is provided, the method including: propagating a neutron beam at the patient, where the neutron beam is emitted from a neutron beam output of a neutron beam converter, where the neutron beam converter is configured in accordance with any embodiment of the first, second, third, fourth, fifth, sixth, seventh, eighth, ninth, and tenth groups of embodiments.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

As used herein and in the appended claims, the singular forms “a,” “an,” and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A neutron beam converter, comprising:
a beam input configured to receive neutrons from a neutron generation target;
a beam output configured to output neutrons;
a central region traversed by an axis between the beam input and the beam output, wherein the central region is configured to scatter neutrons to a treatment energy range;
an intermediate region located laterally around the central region and configured to redirect and scatter neutrons, wherein the intermediate region comprises a first intermediate section and a second intermediate section each comprising a different material; and
a peripheral region located laterally around the intermediate region and configured to absorb neutrons and gamma radiation, wherein the peripheral region comprises a first peripheral section and a second peripheral section each comprising a different material.

2. The converter of claim 1, wherein the central region comprises a first section, a second section, and a third section, wherein each of the first, second, and third sections comprise a different material, and wherein each of the first, second, and third sections are traversed by the axis.

3. The converter of claim 2, wherein the first section comprises fluorine, the second section comprises magnesium, and the third section comprises aluminum.

4. The converter of claim 1, wherein the central region comprises a first material and a second material.

5. The converter of claim 4, wherein the first material comprises aluminum and the second material comprises magnesium.

6. The converter of claim 4, wherein the first material is configured to scatter generated neutrons towards or into a treatment energy range and configured to redirect generated neutrons, and wherein the second material is configured to non-resonantly scatter generated neutrons.

7. The converter of claim 4, wherein the first material comprises lead and fluorine.

8. The converter of claim 4, wherein the second material comprises beryllium.

9. The converter of claim 4, wherein the second material comprises at least one of an oxide, a carbide, or a nitride.

10. The converter of claim 4, wherein the second material is configured as a layer between a first central section comprising the first material and a second central section comprising the first material.

11. The converter of claim 10, wherein the first central section, the layer, and the second central section are traversed by the axis.

12. The converter of claim 4, wherein the central region comprises a third material that comprises at least one of: aluminum, fluorine, and magnesium.

13. The converter of claim 1, wherein the central region comprises a cylindrical portion that extends from the beam input to the beam output.

14. The converter of claim 13, wherein the cylindrical portion has a first terminus upstream of an installed location of the neutron generation target.

15. The converter of claim 13, wherein the cylindrical portion has a first terminus along an upstream-most face of the neutron beam converter.

16. The converter of claim 14, wherein the cylindrical portion has a second terminus in proximity with the beam output.

17. The converter of claim 13, wherein the cylindrical portion is configured to act as a low resistance pathway for neutrons relative to the intermediate region.

18. The converter of claim 13, wherein the cylindrical portion comprises a tubular portion.

19. The converter of claim 18, wherein the cylindrical portion is configured to act as a low resistance pathway for neutrons relative to the intermediate region and a material within the tubular portion.

20. The converter of claim 18, wherein the tubular portion comprises at least one of: aluminum, fluorine, and magnesium, and laterally surrounds a first central section comprising a first material and a second material.

21. The converter of claim 13, wherein the cylindrical portion comprises at least one of: aluminum, fluorine, and magnesium.

22. The converter of claim 13, wherein the cylindrical portion traverses the axis.

23. The converter of claim 1, wherein the converter has a downstream-most surface and a recess from the downstream-most surface at the beam output.

24. The converter of claim 23, wherein the recess has a sidewall portion comprising a neutron redirector.

25. The converter of claim 24, wherein the neutron redirector comprises at least one of lead, nickel, bismuth, and tungsten.

26. The converter of claim 23, wherein the recess is cylindrical and the converter includes a tubular liner comprising beryllium, the tubular liner located in proximity with a sidewall of the recess.

27. The converter of claim 1, wherein the central region comprises:
a first central section traversed by the axis and configured to scatter neutrons to the treatment energy range;
a second central section traversed by the axis, located downstream of the first central section, and configured to scatter neutrons in an epithermal energy range to lower energies; and
a third central section traversed by the axis, located downstream of the second central section, and configured to absorb neutrons scattered to the lower energies.

28. The converter of claim 1, wherein the central region comprises a solid section spaced apart from an installation location of the neutron generation target, wherein the solid section is traversed by the access and located downstream of the installation location of the target.

29. The converter of claim 28, wherein the solid section is spaced apart from the installation location of the target by at least 10 centimeters.

30. The converter of claim 1, wherein the first intermediate section is relatively closer to the central region than the second intermediate section, and wherein the first intermediate section is configured to scatter generated neutrons and the second intermediate section is configured to redirect generated neutrons.

31. The converter of claim 30, wherein the second intermediate section is configured to redirect generated neutrons and absorb photons.

32. The converter of claim 30, wherein the first intermediate section is further configured to redirect neutrons.

33. The converter of claim 32, wherein the first intermediate section further comprises at least one of fluorine, aluminum, and magnesium, and the second intermediate section comprises at least one of lead, nickel, bismuth, and tungsten.

34. The converter of claim 33, wherein the first intermediate section further comprises lead and the second intermediate section comprises lead.

35. The converter of claim 30, wherein the first intermediate section is located laterally around the central region and the second intermediate section is located laterally around the first intermediate section.

36. The converter of claim 35, wherein the first intermediate section is in contact with the central region, and wherein the second intermediate section is in contact with the first intermediate section.

37. The converter of claim 1, wherein the intermediate region extends laterally across a rear face of the central region.

38. The converter of claim 1, wherein the second intermediate section is located laterally around the first intermediate section, and wherein the first intermediate section is configured to scatter neutrons and the second intermediate section is configured to redirect neutrons and absorb gamma radiation.

39. The converter of claim 38, wherein the intermediate region further comprises a third intermediate section located laterally around the second intermediate section, wherein the third intermediate section is configured to scatter neutrons from a first energy in an epithermal energy range to a second energy lower than the first energy.

40. The converter of claim 39, wherein the intermediate region further comprises a fourth intermediate section located laterally around the third intermediate section, wherein the fourth intermediate section is configured to absorb neutrons.

41. The converter of claim 40, wherein the intermediate region further comprises a fifth intermediate section located laterally around the fourth intermediate section, wherein the fifth intermediate section is configured to absorb gamma radiation.

42. The converter of claim 1, wherein the first peripheral section is relatively closer to the intermediate region than the second peripheral section, and wherein the first peripheral section is configured to absorb neutrons and the second peripheral section is configured to absorb photons.

43. The converter of claim 42, wherein the peripheral region further comprises a third peripheral section located relatively closer to the intermediate section than the first peripheral section, wherein the third peripheral section is configured to scatter epithermal neutrons.

44. The converter of claim 42, wherein the peripheral region further comprises a third peripheral section located relatively closer to the intermediate section than the first peripheral section, wherein the third peripheral section is configured to absorb photons.

45. The converter of claim 42, wherein the first and second peripheral sections comprise titanium.

46. The converter of claim 42, wherein the first peripheral section comprises titanium and vanadium.

47. The converter of claim 42, wherein the second peripheral section comprises boron.

48. The converter of claim 42, wherein the first peripheral section is located laterally around the intermediate and central regions and the second peripheral section is located laterally around the first peripheral section.

49. The converter of claim 42, wherein the first peripheral section is in contact with the intermediate region, and wherein the second peripheral section is in contact with the first peripheral section.

50. The converter of claim 42, wherein the peripheral region extends laterally across a portion of the rear of the central region and a portion of the rear of the intermediate region.

51. The converter of claim 1, further comprising a frontal region extending laterally across the converter.

52. The converter of claim 51, wherein the frontal region is configured to absorb neutrons and photons.

53. The converter of claim 52, wherein the frontal region comprises a first frontal section configured to absorb photons and a second frontal section configured to absorb neutrons.

54. The converter of claim 53, wherein the first frontal section is located downstream of the second frontal section.

55. The converter of claim 53, wherein the first frontal section comprises an aperture for a recess at the beam output.

56. The converter of claim 53, wherein the first frontal section comprises at least one of lead, nickel, bismuth, and tungsten.

57. The converter of claim 53, wherein the second frontal section comprises at least one of lithium, cadmium, boron, titanium, gadolinium, indium, hafnium, and a hydrogenous polymer.

58. The converter of claim 53, further comprising a third frontal section configured to scatter epithermal neutrons.

59. The converter of claim 58, wherein the third frontal section is located upstream of the second frontal section.

60. The converter of claim 51, wherein the frontal region forms a continuation of the peripheral region.

61-168. (canceled)
